SEQUENCE LISTING (2 sequences)

SEQ ID NO: 1 - protein, 9 aa, Mus musculus
EGSRNQDWL

SEQ ID NO: 2 - protein, 9 aa, Homo sapiens
KVPRNQDWL

(19) United States
(12) Patent Application Publication
(10) Pub. No.: US 2025/0057949 A1
(43) Pub. Date: Feb. 20, 2025

(54) T CELL THERAPY WITH VACCINATION AS A COMBINATION IMMUNOTHERAPY AGAINST CANCER

(71) Applicant: The United States of America, as represented by the Secretary, Department of Health and Human, Bethesda, MD (US)

(72) Inventors: Sri Krishna, Bethesda, MD (US); Zhiya Yu, Bethesda, MD (US); Ken-ichi Hanada, Bethesda, MD (US); Steven A. Rosenberg, Potomac, MD (US)

(73) Assignee: The United States of America, as represented by the Secretary, Department of Health and Human Servic, Bethesda, MD (US)

(21) Appl. No.: 18/720,347

(22) PCT Filed: Dec. 29, 2022
(86) PCT No.: PCT/US2022/082579

§ 371 Date: Jun. 14, 2024

Related U.S. Application Data
(60) Provisional application No. 63/295,762, filed on Dec. 31, 2021.

(51) Int. Cl. A61K 39/00; A61K 39/395; A61P 35/00

(52) U.S. Cl. CPC A61K 39/464401; A61K 39/0011; A61K 39/3955; A61K 39/4611; A61K 39/4632; A61P 35/00; A61K 2039/54

(57) ABSTRACT

Disclosed are methods of treating or preventing cancer in a mammal, the method comprising: (a) isolating T cells from a tumor sample from the mammal, wherein the isolated T cells are one or both of exhausted and differentiated, and the isolated T cells have antigenic specificity for a tumor-specific antigen expressed by the tumor sample from the mammal, wherein the tumor-specific antigen is a tumor-specific neoantigen or an antigen with a tumor-specific driver mutation; and optionally expanding the numbers of isolated, tumor antigen-specific T cells; and (b) administering to the mammal (i) the isolated T cells of (a) and (ii) a vaccine which specifically stimulates an immune response against the tumor-specific antigen for which the isolated T cells have antigenic specificity.

### CROSS-REFERENCE TO RELATED APPLICATION

This patent application claims the benefit of U.S. Provisional Patent Application No. 63/295,762, filed Dec. 31, 2021, which is incorporated by reference in its entirety herein.

### STATEMENT REGARDING FEDERALLY SPONSORED RESEARCH OR DEVELOPMENT

This invention was made with Government support under project number ZIABC010985 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 2,650 Byte XML file named “766239.xml,” dated Dec. 28, 2022.

### BACKGROUND OF THE INVENTION

Adoptive cell therapy (ACT) using T cells that target a tumor-specific antigen can produce positive clinical responses in some patients. Nevertheless, several obstacles to the successful use of ACT for the treatment of cancer and other conditions remain. For example, exhausted phenotypes of antitumor T cells may be unable to mount a sustained immune response against tumor. Accordingly, there is a need for improved immunotherapies against cancer.

### BRIEF SUMMARY OF THE INVENTION

An aspect of the invention provides a method of treating or preventing cancer in a mammal, the method comprising: (a) isolating T cells from a tumor sample from the mammal, wherein the isolated T cells are one or both of exhausted and differentiated, and the isolated T cells have antigenic specificity for a tumor-specific antigen expressed by the tumor sample from the mammal, wherein the tumor-specific antigen is a tumor-specific neoantigen or an antigen with a tumor-specific driver mutation; and optionally expanding the numbers of isolated, tumor antigen-specific T cells; and (b) administering to the mammal (i) the isolated T cells of (a) and (ii) a vaccine which specifically stimulates an immune response against the tumor-specific antigen for which the isolated T cells have antigenic specificity.

An aspect of the invention provides a method of treating or preventing cancer in a mammal with a tumor, the method comprising: (a) isolating T cells from a biological sample from the mammal with the tumor; (b) introducing into the isolated T cells a nucleic acid comprising a nucleotide sequence encoding an exogenous receptor having antigenic specificity for a tumor-specific antigen expressed by the tumor of the mammal to produce T cells which express the exogenous receptor, wherein the tumor-specific antigen is a tumor-specific neoantigen or an antigen with a tumor-specific driver mutation; and optionally expanding the numbers of T cells which express the exogenous receptor; and (c) administering to the mammal (i) the T cells which express the exogenous receptor of (b) and (ii) a vaccine which specifically stimulates an immune response against the tumor-specific antigen for which the exogenous receptor has antigenic specificity

### BRIEF DESCRIPTION OF THE SEVERAL VIEWS OF THE DRAWING(S)

FIG. 1 is a flowchart showing the steps for neoantigen-specific T cell ACT combined with neoantigen vaccination according to an aspect of the invention.

FIG. 2A is a schematic illustrating the timeline of neoantigen-specific T cell ACT combined with neoantigen vaccination in B16⁺ mice according to an aspect of the invention.

FIG. 2B shows a flow cytometry graph showing the number of Pmel⁺ T cells with expression levels of cluster of differentiation 39 (CD39) categorized into low (CD39^{lo}), medium (CD39^{med}), and high groups (CD39^{hi}) after 2 stimulations.

FIG. 2C shows heat map graphs of PD-1 and TIM3 expression in CD39^{lo} and CD39^{hi} T cells and a graph of 4-1BB activation in CD39^{lo} and CD39^{hi} T cells.

FIGS. 3A-3B are graphs showing the tumor size in mm² of B16⁺ mice over the days post-ACT in mice treated with PBS, bulk Pmel⁺ T cells (Bulk), Pmel⁺ T cells expressing CD39 at high levels (CD39^{hi}), medium levels (CD39^{med}), and low levels (CD39^{lo}) (FIG. 3A) and in PBS, bulk Pmel⁺ T cells (Bulk), CD39^{lo}, CD39^{hi}+isotype control antibody (IgG), CD39^{hi}+anti-CD40 antibody, or CD39^{hi}+anti-PD1 antibody (FIG. 3B).

FIG. 4A is a graph showing the probability of survival of B16⁺ mice over the days post-ACT in groups (n=5) treated with PBS, bulk Pmel⁺ T cells (Bulk Pmel), low expressing CD39 Pmel⁺ T cells (CD39^{lo})+isotype control antibody (Rat IgG), high expressing CD39 Pmel⁺ T cells (CD39^{hi})+anti-CD40 antibody, CD39^{hi}+anti-PD1 antibody, or CD39^{hi}+relevant vaccinia virus against human glycoprotein 100 epitope sequence KVPRNQDWL (SEQ ID NO: 2) (r.VACV_{hgp100}).

FIG. 4B is a graph showing tumor size in mm² of B16⁺ mice over the days post-ACT in mice treated with PBS, Bulk, CD39^{lo}, CD39^{hi}+IgG, CD39^{hi}+anti-CD40 antibody, CD39^{hi}+anti-PD1 antibody, or CD39^{hi}+r.VACV_{hgp100}.

FIGS. 5A-5C are graphs showing tumor size in mm² of B16⁺ mice over the days post-ACT in mice treated with the following, either alone or in various combinations, as shown in the Figure: PBS, low expressing CD39 Pmel⁺ T cells (CD39^{lo}), high expressing CD39 Pmel⁺ T cells (CD39^{hi}), isotype control antibody (IgG), the human glycoprotein 100 peptide (Pep), a vaccinia virus against human glycoprotein 100 (VACV_{hgp100}) (FIG. 5A), an adenovirus against human glycoprotein 100 (ADV_{hgp100}) (FIG. 5B), or an anti-CD40 antibody (FIG. 5C).

FIG. 5D is a graph showing the probability of survival of B16⁺ mice over the days post-ACT treated with low expressing CD39 Pmel⁺ T cells (CD39^{lo}) or high expressing CD39 Pmel⁺ T cells (CD39^{hi}) in combination with (VACV_{hgp100}) or ADV_{hgp100} (n=5).

FIG. 6A is a graph showing tumor size in mm² of B16⁺ mice over the days post-ACT in mice treated with combinations of PBS or low expressing CD39 Pmel⁺ T cells (CD39^{lo}) alone or in combination with the relevant vaccinia virus against human glycoprotein 100 (r.VACVhgp100) (FIG. 6A).

FIG. 6B is a graph showing tumor size in mice treated with PBS or PD1 and TIM3 expressing Pmel⁺ T cells (PD1⁺TIM3⁺) alone or in combination with r.VACVhgp100.

FIG. 6C is a graph showing tumor size in mice treated with PBS or CD39 and CD69 expressing Pmel⁺ T cells (CD39⁺CD69⁺) alone or in combination with r.VACVhgp100.

FIGS. 7A-7B are graphs showing tumor size in mm² of B16⁺ mice over the days post-ACT in mice treated with PBS or CD39 Pmel⁺ T cells (CD39^{lo}) alone or in combination with a vaccinia virus against the irrelevant HLA-A2 restricted human glycoprotein 100 (hgp100) epitope (VACV_{hgp100(209).irr}) or a vaccinia virus against the relevant hgp100 epitope 25 (hgp100₂₅) (VACV_{hgp100(25)}) (FIG. 7A) or CD39 and CD69 expressing Pmel⁺ T cells (CD39⁺CD69⁺) alone or in combination with VACV_{hgp100(209).irr} or VACV_{hgp100(25)} (FIG. 7B).

FIGS. 8A-8F are bar graphs showing the percentage of Thy1.1⁺ Vβ13⁺ CD8⁺ T cells over total CD8⁺ T cells at days 3, 7, and 10 post-ACT in the spleen (FIG. 8A), the draining lymph node (LN) (FIG. 8B), and the tumor (FIG. 8C) of B16⁺ mice treated with either low expressing CD39 Pmel⁺ T cells (CD39^{lo}), CD39^{lo} and relevant vaccinia virus against human glycoprotein 100 (rVACVhgp100), or just rVACV_{hgp100}, and bar graphs showing the percentage of Thy1.1⁺ Vβ13⁺ CD8⁺ T cells over total CD8⁺ T cells at days 3, 7, and 10 post-ACT in the spleen (FIG. 8D), the draining LN (FIG. 8E), and the tumor (FIG. 8F) of B16⁺ mice treated with either CD39 and CD69 expressing Pmel⁺ T cells (CD39⁺CD69⁺), CD39⁺CD69⁺ and rVACV_{hgp100}, or just rVACV_{hgp100}.

FIG. 9 shows three bar graphs showing the percentage of terminally exhausted PD1⁺TIM3⁺ adoptively transferred Thy1.1⁺Vβ13⁺ neoantigen-specific T cells isolated from tumors at day 3, 7, and 10 post-ACT in B16⁺ mice treated with either low expressing CD39 Pmel⁺ T cells (CD39^{lo}), CD39^{lo} and relevant vaccinia virus against human glycoprotein 100 (rVACVhgp100), CD39 and CD69 expressing Pmel⁺ T cells (CD39⁺CD69⁺), or CD39⁺CD69⁺ and rVACV_{hgp100}.

FIG. 10 is a graph showing tumor size in mm² of B16⁺ mice over the days post-ACT in mice treated with PBS or CD39 and CD69 expressing Pmel⁺ T cells (CD39⁺CD69⁺) alone or in various combinations with one or more of irrelevant peptide influenza nucleoprotein (Flu.NP), a vaccinia virus against the relevant hgp100 epitope 25 (hgp100₂₅) (VACV_{hgp100(25)}), anti-CD40 antibody, and isotype control antibody (Rat IgG).

FIG. 11 is a graph showing tumor size in mm² of B16⁺ mice over the days post-ACT in mice treated with combinations of PBS or Pmel⁺ T cells alone or in combination with bone marrow derived dendritic cells (DC) loaded with irrelevant influenza peptide (Irr.Pep) or relevant neoepitope (hgp100^{KVP}).

FIGS. 12A-12C are graphs showing MC38 tumor size in mm² in C57BL/6 mice over the days post-ACT in mice treated with either PBS alone or in combination with a vaccinia virus against hgp100 (VACV_{hgp100}) (FIG. 12A), or low expressing CD39 Pmel⁺ T cells (CD39^{lo}) alone or in combination with VACV_{hgp100} (FIG. 12B), or CD39 and CD69 expressing Pmel⁺ T cells (CD39⁺CD69⁺) alone or in combination with VACV_{hgp100} (FIG. 12C).

FIGS. 13A-13B are graphs showing tumor size in mm² of β2M knock-out (KO) mice over the days post-ACT in mice treated with PBS alone or in combination a vaccinia virus against the relevant hgp100₂₅ (VACV_{hgp100(25)}), and either low expressing CD39 Pmel⁺ T cells (CD39^{lo}) alone or in combination with VACV_{hgp100(25)} (FIG. 13A), or CD39 and CD69 expressing Pmel⁺ T cells (CD39⁺CD69⁺) alone or in combination with VACV_{hgp100(25)} (FIG. 13B).

FIGS. 14A-14B are graphs showing B16KVP tumor size in mm² of C57BL/6 mice over the days post-ACT in mice treated with of the following, either alone or in various combinations, as shown in the Figures: PBS, anti-B7.1 and anti-B7.2 antibodies (anti-B7.1/2), isotype control antibody (IgG), a vaccinia virus against the relevant hgp100₂₅ (VACV_{hgp100(25)}), and either low expressing CD39 Pmel⁺ T cells (CD39^{lo}) (FIG. 14A), or CD39 and CD69 expressing Pmel⁺ T cells (CD39⁺CD69⁺) (FIG. 14B).

FIG. 15A is a flow chart illustrating the patient's course of treatment.

FIG. 15B shows flow cytometry graphs showing the frequencies of HLA-A0201-restricted GP100 tetramers as a percentage of CD8⁺ TILs within the infusion product (Rx1 and Rx3) showing no apparent differences in GP100 TIL frequency administered to the patient during the course of the first intravenous treatment without the vaccine (Rx1) and the second intravenous treatment in conjunction with the GP100 vaccine (Rx3).

FIG. 15C shows a scatter plot showing the clonal frequencies of the GP100 TCR within Rx1 and Rx3, showing no apparent differences in the clonal distribution of the treatment product, with the immunodominant GP100 TCR labeled.

FIGS. 15D-15G show heatmap graphs of GP100 TILs within the infusion product (Rx1 and Rx3) showing no apparent differences in the phenotypic state of GP100 TIL frequency administered to the patient during the course of Rx1 and Rx3 with plots showing expression of CD39 and CD69 (FIG. 15D), CD62L and CD8 (FIG. 15E), TIM3 and CD8 (FIG. 15F), and CD39 and CD8 (FIG. 15G). Thus both Rx1 and Rx3 included similarly differentiated dysfunctional antitumor TIL that was able to elicit tumor regression only with the vaccine administered at the time of Rx3 but not in the absence of vaccine (Rx1).

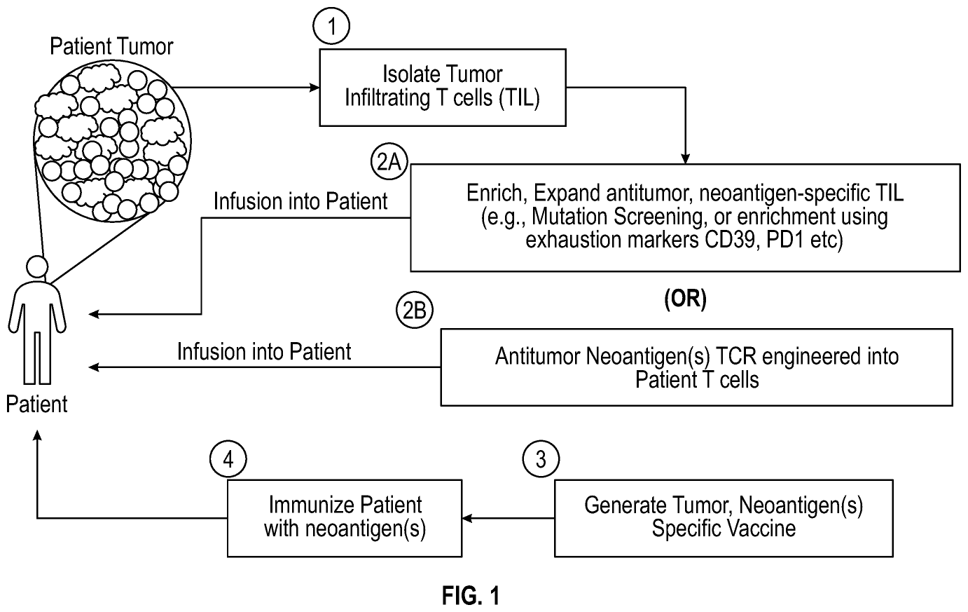

### DETAILED DESCRIPTION OF THE INVENTION

ACT against cancer involves in vitro expansion and in vivo administration of autologous antitumor T-cells targeting patient's own tumors. Antitumor T cells, e.g., those targeting tumor-specific mutations (“neoantigens”), may exist in a terminally differentiated, exhausted state. These exhausted antitumor T-cells may have limited efficacy during ACT against established tumors, and may have limited persistence in vivo in patients post-treatment. Conventional cell therapies using antitumor T cells may not provide significant responses. It has been discovered that combination immunotherapy comprising (i) T cells that are one or both of exhausted and differentiated and which have antigenic specificity for a tumor-specific antigen and (ii) a vaccine which specifically stimulates an immune response against the tumor-specific antigen can result in superior antitumor treatment in vivo. Administration of a vaccine which specifically stimulates an immune response against the tumor-specific antigen can enhance ACT using T cells that are one or both of exhausted and differentiated and which have antigenic specificity for the same tumor-specific antigen. The antitumor effect of this combination immunotherapy may be superior to that mediated by vaccine alone or ACT using T cells that are one or both of exhausted and differentiated alone. Thus, ACT using T cells that are one or both of exhausted and differentiated, or gene-engineered (TCR-transduced T cells) can be synergistically enhanced using a vaccine which specifically stimulates an immune response against the same tumor-specific antigen. The inventive methods may, advantageously, overcome the challenge(s) to developing an effective immunotherapy using antitumor T cells with an exhausted phenotype, which may otherwise be unable to mount a sustained immune response against the tumor.

The inventive methods may, advantageously, target metastatic cancers, such as epithelial cancers that cause more than about 90% of all cancer deaths. These cancers may not respond to conventional immunotherapies and may not respond to antitumor vaccines as single agents.

By combining a vaccine with the administration of T cells that are one or both of exhausted and differentiated, the inventive methods may rescue antitumor activity of exhausted and/or differentiated T cells. In this regard, an aspect of the invention provides a method of treating or preventing cancer in a mammal. The method may comprise isolating T cells from a tumor sample from the mammal. The tumor sample may be, for example, tissue from primary tumors or tissue from the site of metastatic tumors. As such, the tumor sample may be obtained by any suitable means, including, without limitation, aspiration, biopsy, or resection.

The isolated T cells may be one or both of exhausted and differentiated. T cell exhaustion is a state of T cell dysfunction that arises in response to chronic antigen stimulation. It is defined by poor effector function, sustained expression of inhibitory receptors and a phenotype that is distinct from that of functional effector or memory T cells. The poor effector function typically exhibited by exhausted T cells may be ameliorated or overcome in the inventive methods by combining the exhausted T cells with a vaccine that targets the same antigen targeted by the exhausted T cells, as described herein. In an aspect of the invention the isolated T cells express any one or more of the following markers of T cell exhaustion: (a) RNA encoding any one or more of: 4-1BB⁺, CCL3+, CD28⁻, CD39⁺, CD62L⁻ (SELL⁻), CD69⁺, CTLA4⁺, CX3CR1⁺, CXCL13⁺, CXCR6⁺, GZMA⁺, GZMB⁺, GZMK⁺, IL7R⁻, LAG-3⁺, LAYN⁺, LEF1⁻, PD-1⁺, PRF1⁺, TCF7⁻, TIGIT⁺, TIM-3⁺, and TOX⁺; and (b) any one or more of the following proteins: 4-1BB⁺, CCL3⁺, CD28⁻, CD39⁺, CD62L⁻ (SELL⁻), CD69⁺, CTLA4⁺, CX3CR1⁺, CXCL13⁺, CXCR6⁺, GZMA⁺, GZMB⁺, GZMK⁺, IL7R⁻, LAG-3⁺, LAYN⁺, LEF1⁻, PD-1⁺, PRF1⁺, TCF7⁻, TIGIT⁺, TIM-3⁺, and TOX⁺. As used herein, the symbol “⁺,” with reference to expression of the indicated marker of T cell exhaustion, encompasses “high” (“^{hi}”) and “medium” (“^{med}”) expression of the indicated marker of T cell exhaustion, and means that the cell upregulates expression of the indicated marker as compared to less exhausted T cells. Upregulated expression may encompass, for example, a quantitative increase in expression of the indicated marker of T cell exhaustion by an average logarithmic fold change (to the base 2) of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, or a range of any two of the foregoing values, or more. As used herein, the symbol “⁻,” with reference to expression of the indicated marker of T cell exhaustion, encompasses a lack of expression and “low” (“^{lo}”) expression of the indicated marker, and means that the cell downregulates expression of the indicated marker as compared to less exhausted T cells. Downregulated expression may encompass, for example, a quantitative decrease in expression of the indicated marker of T cell exhaustion by an average logarithmic fold change (to the base 2) of about −1, about −2, about −3, about −4, about −5, about −6, about −7, about −8, about −9, about −10, about −20, about −30, about −40, about −50, about −60, about −70, about −80, about −90, about −100, about −110, about −120, about −130, about −140, about −150, about −160, about −170, about −180, about −190, about −200, about −210, about −220, about −230, about −240, about −250, about −260, about −270, about −280, about −290, about −300, about −310, about −320, about −330, about −340, about −350, about −360, about −370, about −380, about −390, about −400, about −410, about −420, about −430, about −440, about −450, about −460, about −470, about −480, about −490, about −500, about −510, about −520, about −530, about −540, about −550, about −560, about −570, about −580, about −590, about −600, or a range of any two of the foregoing values, or more. Isotype controls can be used to distinguish marker expression. Within the gate for a given marker, the bottom tertile expression can be designated as “^{lo},” the middle tertile can be designated as “^{med}” and the upper tertile can be designated as “^{hi}.”

T cell differentiation refers to the process by which a precursor cell acquires characteristics of a more mature T-cell. T-cell differentiation follows a linear progression along a continuum of major clusters (e.g., progressing in the following order: naïve T cell (T_{N}), T memory stem cell (T_{SCM}), central memory T cell (T_{CM}), effector memory T cell (T_{EM}), and terminal effector (T_{TE}) cells), where less differentiated cells give rise to more differentiated progeny in response to antigenic stimulation. With increasing differentiation, memory T cells progressively acquire or lose specific functions. For example, the increased differentiation of T cells is believed to negatively affect the capacity of T cells to function in vivo. The poor effector function typically exhibited by differentiated T cells may be ameliorated or overcome in the inventive methods by combining the differentiated T cells with a vaccine that targets the same antigen targeted by the differentiated T cells, as described herein. In an aspect of the invention, the isolated T cells are terminally differentiated.

In an aspect of the invention, the isolated T cells express any one or more of the following markers of differentiation: (a) RNA encoding any one or more of: CCR7⁻, CD27⁻, CD45RA⁺, CD45RO⁻, CD95⁺, EOMES⁻, FOXO1⁻, KLRG1⁺, T-BET⁺, TCF7⁻, TOX⁺, and ZEB2⁺; and (b) any one or more of the following proteins: CCR7⁻, CD27⁻, CD45RA⁺, CD45RO⁻, CD95⁺, EOMES⁻, FOXO1⁻, KLRG1⁺, T-BET⁺, TCF7⁻, TOX⁺, and ZEB2⁺. As used herein, the symbol “⁺,” with reference to expression of the indicated marker of T cell differentiation, encompasses “high” (“^{hi}”) and “medium” (“^{med}”) expression of the indicated marker of T cell differentiation, and means that the cell upregulates expression of the indicated marker as compared to less differentiation T cells. Upregulated expression may encompass, for example, a quantitative increase in expression of the indicated marker of T cell differentiation by an average logarithmic fold change (to the base 2) of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, or a range of any two of the foregoing values, or more. As used herein, the symbol “⁻,” with reference to expression of the indicated marker of T cell differentiation, encompasses a lack of expression and “low” (“^{lo}”) expression of the indicated marker, and means that the cell downregulates expression of the indicated marker as compared to less differentiated T cells. Downregulated expression may encompass, for example, a quantitative decrease in expression of the indicated marker of T cell differentiation by an average logarithmic fold change (to the base 2) of about −1, about −2, about −3, about −4, about −5, about −6, about −7, about −8, about −9, about −10, about −20, about −30, about −40, about −50, about −60, about −70, about −80, about −90, about −100, about −110, about −120, about −130, about −140, about −150, about −160, about −170, about −180, about −190, about −200, about −210, about −220, about −230, about −240, about −250, about −260, about −270, about −280, about −290, about −300, about −310, about −320, about −330, about −340, about −350, about −360, about −370, about −380, about −390, about −400, about −410, about −420, about −430, about −440, about −450, about −460, about −470, about −480, about −490, about −500, about −510, about −520, about −530, about −540, about −550, about −560, about −570, about −580, about −590, about −600, or a range of any two of the foregoing values, or more. Isotype controls can be used to distinguish marker expression, as described herein with respect to other aspects of the invention.

The isolated T cells may have antigenic specificity for a tumor-specific antigen expressed by the tumor sample from the mammal. The phrases “antigen-specific” and “antigenic specificity,” as used herein, mean that the T cell can specifically bind to and immunologically recognize an antigen, or an epitope thereof, such that binding of the T cell to the antigen, or the epitope thereof, elicits an immune response.

The term “tumor-specific antigen” or “tumor antigen,” as used herein, refers to any molecule (e.g., protein, polypeptide, peptide, lipid, carbohydrate, etc.) solely or predominantly expressed or over-expressed by a tumor cell, such that the antigen is associated with the tumor. The tumor-specific antigen can additionally be expressed by normal, non-tumor, or non-cancerous cells. However, in such cases, the expression of the tumor-specific antigen by normal, non-tumor, or non-cancerous cells is not as robust as the expression by tumor. In this regard, the tumor cells can over-express the antigen or express the antigen at a significantly higher level, as compared to the expression of the antigen by normal, non-tumor, or non-cancerous cells. Also, the tumor-specific antigen can additionally be expressed by cells of a different state of development or maturation. For instance, the tumor-specific antigen can be additionally expressed by cells of the embryonic or fetal stage, which cells are not normally found in an adult host. Alternatively, the tumor-specific antigen can be additionally expressed by stem cells or precursor cells, which cells are not normally found in an adult host.

In an aspect of the invention, the tumor-specific antigen may be a tumor-specific neoantigen. Neoantigens are a class of tumor-specific antigen which arise from cancer-specific mutations in expressed protein. The term “neoantigen” relates to a peptide or protein expressed by a tumor cell that includes one or more amino acid modifications compared to the corresponding wild-type (non-mutated) peptide or protein that is expressed by a normal (non-cancerous) cell. A neoantigen may be patient-specific. In an aspect of the invention, the tumor-specific neoantigen is a personal neoantigen encoded by one or more somatic mutation(s) that are unique to the mammal's tumor, optionally wherein the tumor-specific neoantigen is not a tumor-specific driver mutation.

In an aspect of the invention, the tumor-specific antigen may be an antigen with a tumor-specific driver mutation. A tumor-specific driver mutation is a mutation that is found in tumor cells, but not in normal (non-cancerous) cells, and which induces cell proliferation and tumor growth. Driver mutations confer a growth advantage on the tumor cells carrying them. Examples of tumor-specific driver mutations include, but are not limited to, mutated ALK, mutated APC, mutated ATRX, mutated BRAF, mutated CDKN2A, mutated DDX3X, mutated DNMT3A, mutated EGFR, mutated ESR1, mutated EWSR1, mutated FGFR1, mutated FLI1, mutated HRAS, mutated IDH1, mutated IDH2, mutated KMT2C, mutated KRAS, mutated MYC, mutated NOTCH1, mutated NRAS, mutated PIK3CA, mutated PTCH1, mutated PTEN, mutated RB1, mutated RUNX1, mutated SETD2, mutated SMARCA4, mutated STK11, and mutated TP53.

In an aspect of the invention, the method comprises screening the tumor for expression of the tumor-specific antigen. Methods of screening tumors for expression of antigens are known in the art. For example, screening the tumor for expression of the tumor-specific antigen may comprise sequencing the whole exome, the whole genome, or the whole transcriptome of a cell of the tumor. Sequencing may be carried out in any suitable manner known in the art. Examples of sequencing techniques that may be useful in the inventive methods include Next Generation Sequencing (NGS) (also referred to as “massively parallel sequencing technology”) or Third Generation Sequencing.

In an aspect of the invention, the method optionally comprises expanding the numbers of isolated, tumor antigen-specific T cells. Expansion of the numbers of T cells can be accomplished by any of a number of methods as are known in the art as described in, for example, U.S. Pat. Nos. 8,034,334; 8,383,099; 11,401,503; Dudley et al., J. Immunother., 26:332-42 (2003); and Riddell et al., J. Immunol. Methods, 128:189-201 (1990). For example, expansion of the numbers of T cells is carried out by culturing the T cells with OKT3 antibody, IL-2, and feeder PBMC (e.g., irradiated allogeneic PBMC). In an aspect of the invention, the method further comprises expanding the numbers of isolated, tumor antigen-specific T cells.

The method may comprise administering to the mammal (i) the isolated T cells and (ii) a vaccine which specifically stimulates an immune response against the tumor-specific antigen for which the isolated T cells have antigenic specificity. In an aspect of the invention, the method comprises administering the T cells to the mammal intravenously or intraperitoneally. In an aspect of the invention, the isolated T cells are tumor infiltrating lymphocytes (TIL). In an aspect of the invention, the isolated T cells are CD4⁺. In another aspect of the invention, the isolated T cells are CD8⁺.

The method may comprise administering a pharmaceutical composition comprising the isolated T cells and a pharmaceutically acceptable carrier. The pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In an embodiment, the pharmaceutically acceptable carrier is supplemented with human serum albumin.

The vaccine may be any type of vaccine that specifically stimulates an immune response against the tumor-specific antigen. Examples of vaccines include, but are not limited to, a cancer cell vaccine, a conjugate polysaccharide vaccine, a dendritic cell vaccine, a DNA vaccine, an inactivated vaccine (any type), a live-attenuated vaccine, a nanoparticle vaccine, a peptide vaccine, a protein vaccine, a recombinant vaccine, an RNA vaccine, a subunit vaccine, and a viral vaccine. Examples of viral vaccines include, but are not limited to, an adenovirus (ADV) vaccine, a vaccinia virus (VACV) vaccine, and a fowl pox virus vaccine.

In an aspect of the invention, the method comprises administering no more than a single dose of the vaccine to the mammal. In other aspects of the invention, the method comprises administering two, three, or more doses of the vaccine to the mammal. In an aspect of the invention, the method comprises administering the vaccine to the mammal every other day starting on a first day that the T cells are administered to the mammal. In aspects, the method may comprise administering the vaccine to the mammal intramuscularly, subcutaneously, intravenously, or intraperitoneally.

In an aspect of the invention, the method further comprises administering an adjuvant to the mammal. An adjuvant may enhance the magnitude and durability of the immune response against the tumor-specific antigen. In an aspect of the invention, the adjuvant comprises an anti-CD40 antibody or an anti-PD-1 antibody.

In an aspect of the invention, the method comprises administering the isolated T cells and the vaccine which specifically stimulates an immune response against the tumor-specific antigen within 30 days of each other. In an aspect of the invention, the method may comprise administering the isolated T cells and the vaccine within 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, or 1 day of each other. For example, the method may comprise administering the isolated T cells and the vaccine within 48 hours of each other, 36 hours of each other, 24 hours of each other, or 12 hours of each other. In an aspect of the invention, the method may comprise administering the vaccine which specifically stimulates an immune response against the tumor-specific antigen within 14 days, 13 days, 12 days, 11 days, 10 days, 9 days, 8 days, 7 days, 6 days, 5 days, 4 days, 3 days, 2 days, or 1 day before administering the isolated T cells. In an aspect of the invention, the method may comprise administering the vaccine which specifically stimulates an immune response against the tumor-specific antigen within 48 hours, 36 hours, 24 hours, or 12 hours before administering the isolated T cells.

In an aspect of the invention, the isolated T cells and the vaccine are administered to the mammal simultaneously. In an aspect of the invention, the isolated T cells and the vaccine are administered to the mammal together in the same composition. In an aspect of the invention, the isolated T cells and the vaccine are administered to the mammal simultaneously but separately.

In an aspect of the invention, the isolated T cells and the vaccine are administered to the mammal sequentially. For example, the isolated T cells may be administered to the mammal before the vaccine is administered to the mammal. In an aspect of the invention, the isolated T cells are administered to the mammal within 24 hours before the vaccine is administered to the mammal. In an aspect of the invention, the vaccine is administered to the mammal before the isolated T cells are administered to the mammal. For example, the isolated T cells may be administered to the mammal within 24 hours after the vaccine is administered to the mammal.

The inventive methods may, advantageously, also augment the antitumor activity of T cells that are not necessarily one or both of exhausted and differentiated. For example, the inventive methods may also augment the antitumor activity of T cells that have been modified to express an exogenous receptor, but are not necessarily one or both of exhausted and differentiated.

An aspect of the invention provide a method of treating or preventing cancer in a mammal with a tumor. The method may comprise isolating T cells from a biological sample from the mammal with the tumor. In an aspect of the invention, the biological sample is a sample of the tumor. The tumor sample may be as described herein with respect to other aspects of the invention. The isolated T cells may be TIL. In an aspect of the invention, the biological sample is a peripheral blood sample.

While, in aspects of the invention, the T cells isolated from the biological sample may be one or both of exhausted and differentiated, preferably, the T cells isolated from the biological sample are not exhausted or differentiated and may, instead have a less differentiated phenotype.

The method may comprise introducing into the isolated T cells a nucleic acid comprising a nucleotide sequence encoding an exogenous receptor having antigenic specificity for a tumor-specific antigen expressed by the tumor of the mammal to produce T cells which express the exogenous receptor. By “exogenous” is meant that the receptor is not native to (naturally-occurring on) the T cell. The tumor-specific antigen may be a tumor-specific neoantigen or an antigen with a tumor-specific driver mutation. The antigenic specificity and the tumor-specific antigen may be as described herein with respect to other aspects of the invention.

In an aspect of the invention, the exogenous receptor having antigenic specificity for the tumor-specific antigen is a T cell receptor (TCR). The exogenous TCR may be a recombinant TCR. A recombinant TCR is a TCR which has been generated through recombinant expression of one or more exogenous TCR α-, β-, γ-, and/or δ-chain encoding genes. A recombinant TCR can comprise polypeptide chains derived entirely from a single mammalian species, or the recombinant TCR can be a chimeric or hybrid TCR comprised of amino acid sequences derived from TCRs from two different mammalian species. For example, the antigen-specific TCR can comprise a variable region derived from a human TCR, and a constant region of a murine TCR such that the TCR is “murinized.” Any exogenous TCR having antigenic specificity for a tumor-specific antigen may be useful in the inventive methods. The TCR generally comprises two polypeptides (i.e., polypeptide chains), such as an α-chain of a TCR, a β-chain of a TCR, a γ-chain of a TCR, a δ-chain of a TCR, or a combination thereof. Such polypeptide chains of TCRs are known in the art. The TCR can comprise any amino acid sequence, provided that the TCR can specifically bind to and immunologically recognize a tumor-specific antigen or epitope thereof. Examples of exogenous TCRs that may be useful in the inventive methods include, but are not limited to, those disclosed in, for example, U.S. Pat. Nos. 7,820,174; 7,915,036; 8,088,379; 8,216,565; 8,431,690; 8,613,932; 8,785,601; 9,128,080; 9,345,748; 9,487,573; 9,879,065; 11,306,131 and U.S. Patent Application Publication Nos. 2013/0116167, each of which is incorporated herein by reference.

In an embodiment of the invention, the exogenous receptor is a chimeric antigen receptor (CAR). Typically, a CAR comprises the antigen binding domain of an antibody, e.g., a single-chain variable fragment (scFv), fused to the transmembrane and intracellular domains of a TCR. Thus, the antigenic specificity of a CAR can be encoded by a scFv which specifically binds to the cancer antigen, or an epitope thereof. Any CAR having antigenic specificity for a tumor-specific antigen may be useful in the inventive methods. Examples of CARs that may be useful in the inventive methods include, but are not limited to, those disclosed in, for example, U.S. Pat. Nos. 8,465,743; 9,266,960; 9,765,342; 9,359,447; 9,868,774; and 10,287,350, each of which is incorporated herein by reference.

The nucleic acid comprising a nucleotide sequence encoding the exogenous receptor may be introduced into the isolated T cells by any suitable technique such as, e.g., gene editing, transfection, transformation, or transduction as described, for example, Green and Sambrook, Molecular Cloning: A Laboratory Manual (4^{th} Ed.), Cold Spring Harbor Laboratory Press (2012). Many transfection techniques are known in the art and include, for example, calcium phosphate DNA co-precipitation; DEAE-dextran; electroporation; cationic liposome-mediated transfection; tungsten particle-facilitated microparticle bombardment; and strontium phosphate DNA co-precipitation. Phage or viral vectors can be introduced into T cells, after growth of infectious particles in suitable packaging cells, many of which are commercially available.

The method may optionally comprise expanding the numbers of T cells which express the exogenous receptor. Expanding the numbers of T cells may be carried out as described herein with respect to other aspects of the invention. In an embodiment of the invention, the method comprises expanding the numbers of T cells which express the exogenous receptor.

The method further comprises administering to the mammal (i) the T cells which express the exogenous receptor and (ii) a vaccine which specifically stimulates an immune response against the tumor-specific antigen for which the exogenous receptor has antigenic specificity. The vaccine may be as described herein with respect to other aspects of the invention.

The method may comprise administering (i) the T cells which express the exogenous receptor and (ii) the vaccine to the mammal within 30 days of each other. In an aspect of the invention, the method may comprise administering the T cells which express the exogenous receptor and the vaccine within 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, or 1 day of each other. For example, the method may comprise administering the T cells which express the exogenous receptor and the vaccine within 48 hours of each other, 36 hours of each other, 24 hours of each other, or 12 hours of each other. The administering of the T cells and the administering of the vaccine may be as described herein with respect to other aspects of the invention. In an aspect of the invention, the method may comprise administering the vaccine which specifically stimulates an immune response against the tumor-specific antigen within 14 days, 13 days, 12 days, 11 days, 10 days, 9 days, 8 days, 7 days, 6 days, 5 days, 4 days, 3 days, 2 days, or 1 day before administering the T cells which express the exogenous receptor. In an aspect of the invention, the method may comprise administering the vaccine which specifically stimulates an immune response against the tumor-specific antigen within 48 hours, 36 hours, 24 hours, or 12 hours before administering the T cells which express the exogenous receptor.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount or any level of treatment or prevention of cancer in a mammal. Furthermore, the treatment or prevention provided by the inventive method can include treatment or prevention of one or more cancers or symptoms of the cancer being treated or prevented. For example, treatment or prevention can include promoting the regression of a tumor. Also, for purposes herein, “prevention” can encompass delaying the onset of the cancer, or a symptom, condition, or recurrence thereof.

The cancer may, advantageously, be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, cholangiocarcinoma, chronic lymphocytic leukemia, chronic myeloid cancer, colon cancer, esophageal cancer, uterine cervical cancer, gastric cancer, gastrointestinal carcinoid tumor, glioma, Hodgkin lymphoma, hypopharynx cancer, kidney cancer, larynx cancer, liver cancer, lung cancer (e.g., non-small cell lung cancer), malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, urinary bladder cancer, solid tumors, and liquid tumors. Preferably, the cancer is an epithelial cancer. In an embodiment, the cancer is cholangiocarcinoma, melanoma, colon cancer, rectal cancer, breast cancer, lung cancer, anal cancer, esophageal cancer, or gastric cancer. Preferably, the cancer expresses the tumor-specific antigen. In an aspect of the invention, the cancer is a virus-associated cancer. Virus-associated cancers include, but are not limited to, HBV+, HCV+, HIV+, HPV+, HTLV+, HHV8+, MCPyV+, and EBV+-associated cancers.

The mammal referred to in the inventive methods can be any mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). Preferably, the mammals are from the order Artiodactyla, including Bovines (cows) and Swines (pigs) or of the order Perssodactyla, including Equines (horses). Preferably, the mammals are of the order Primates, Ceboids, or Simoids (monkeys) or of the order Anthropoids (humans and apes). A more preferred mammal is the human. In an especially preferred embodiment, the mammal is the patient expressing the tumor-specific antigen.

The following examples further illustrate the invention but, of course, should not be construed as in any way limiting its scope.

### EXAMPLES

The following materials and methods were employed in the experiments described in Examples 1-13.

### Mouse and Tumor Lines

The B16 melanoma murine tumor line was modified by inserting the human GP100 epitope sequence at its murine counterpart, making it a neoepitope that can be targeted by Pmel TCR transgenic mouse T cells (EGSRNQDWL (SEQ ID NO: 1)->KVPRNQDWL (SEQ ID NO: 2)) (Hanada et al., JCI Insight, 4(10): e124405 (2019)).

Mice from the Pmel mouse line, which express T cell receptors recognizing peptide epitope 25-33 derived from both mouse and human gp100, was used to obtain exhausted T cells. Mouse tumor line B16KVP (or “B16”) (a human melanoma model) and the MC38KVP (or “MC38”) mouse tumor line (a human colon cancer model) were used to test the efficacy of treatments on tumor progression. Both B16 and MC38 were derived from C57BL/6 background mice and express an antigenic epitope that can be recognized by Pmel-1 T cells. Wildtype C57BL/6 and b2M KO mice were used in tumor treatment experiments.

The B16 and MC38 cell lines were cultured in complete medium (CM): RPMI 1640 with 10% FBS, 1 mM sodium pyruvate (Thermo Fisher Scientific), 1× Non-Essential Amino Acids (Thermo Fisher Scientific), 55 μM 2-mercaptoethanol (Gibco), 1× antibiotic-antimycotic (Thermo Fisher Scientific), and 50 μg/ml gentamicin (Gibco). Modified B16 cell lines were maintained in CM with blasticidin S (10 μg/ml; Invivogen) or puromycin (5 μg/ml; Invivogen).

### Cell Culturing

Pmel TCR transgenic mice were used as T cells for ACT. Splenocytes from the mice were isolated by single cell suspension. Hgp100 neoepitope (KVPRNQDWL) (SEQ ID NO: 2) was added at 10 ug/mL concentration to the cell suspension and cultured in CM with 30 IU/ml recombinant human IL-2 (rhIL-2; Prometheus Laboratories). On day 5, cells were split and restimulated using anti-CD3 and anti-CD28 antibodies in 24 well plates. On day 10 (day of treatment), exhausted T cells were obtained from Pmel mice and sorted by FACS based on CD39 expression, high CD39 and CD69 co-expression, or PD1 and TIM3 co-expression. Less-exhausted T cells were obtained by sorting for CD39^{lo} and negative T cells, or by sorting for CD39/CD69 low expressing T cells.

### Adoptive Cell Therapy

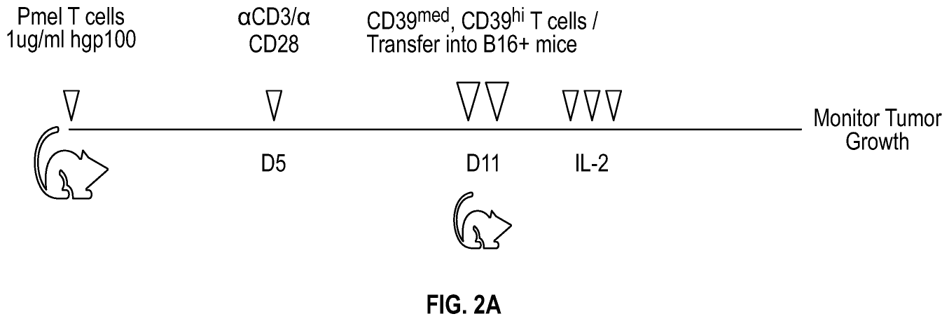

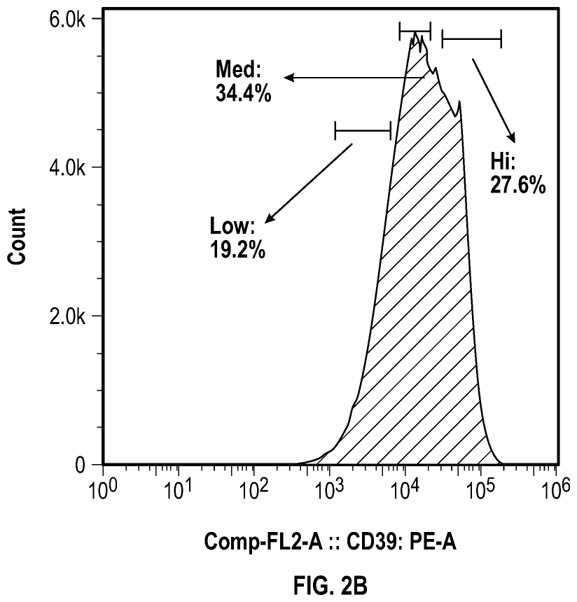

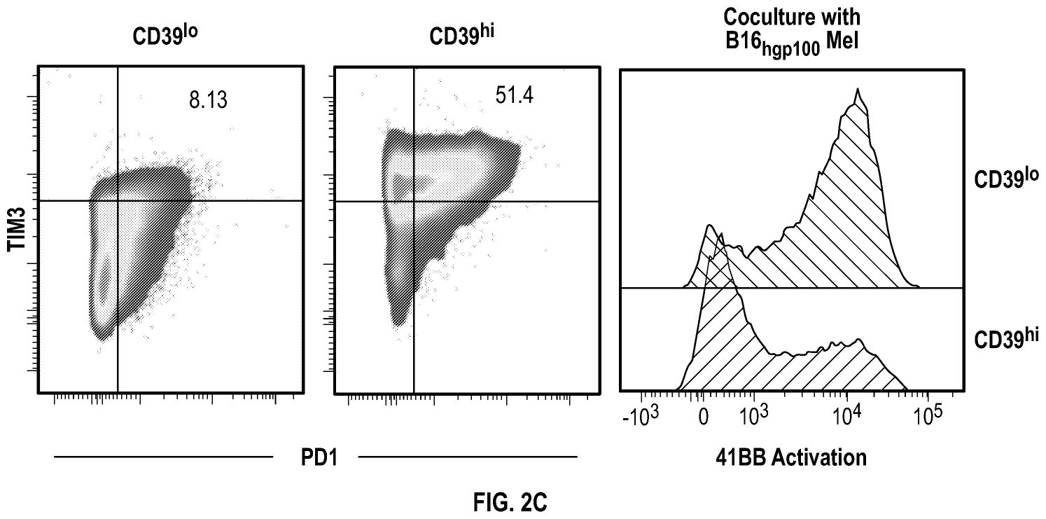

Adoptive cell therapy (ACT) was performed as shown in FIG. 2A. A Pmel mouse was used to obtain exhausted T cells and either B16⁺, MC38, or β2M KO mice with tumors were used as recipients. Mice were subcutaneously injected with 5×10⁵ tumor cells. Ten days later, tumor-bearing mice received 5 Gy of total-body irradiation. On day 11, after tumor inoculation, mice were treated with cultured exhausted or less-exhausted Pmel T cells (1E6 T cells per mice) i.v. with or without 2×10⁷ PFU of recombinant human gp100-vaccinia (rVVhgp100), or using 1E8 PFU recombinant adenovirus expressing the hgp100 neoepitope, or using peptide vaccinations (i.p. or i.v.) along with anti-CD40 agonistic antibody (i.p. or i.v.). In addition, 180,000 IU of rhIL-2 was injected i.p. into mice daily for 3 days after cell transfer.

### ACT Complementary Vaccination or Immunomodulator Treatment

In combination with ACT, mice were injected with combinations of PBS, vaccines, antibodies, and peptides, as shown in FIG. 2A. The neoantigen vaccines used were a vaccinia virus against human glycoprotein 100 (r.VACV_{hgp100}), a vaccinia virus against human glycoprotein 100 amino acids 25-33 epitope (VACV_{hgp100(25)}), a vaccinia virus against the irrelevant HLA-A2 restricted hgp100 epitope (VACV_{hgp100(209)irr.}), an adenovirus against human glycoprotein 100 (ADV_{hgp100}), a bone marrow derived dendritic cell (BMDC) loaded via four hours of peptide pulsing with either the relevant human glycoprotein 100 amino acids 25-33 epitope (DC+hgp100^{KVP} Pep) or the irrelevant influenza virus peptide (DC+Irr.Pep) which were injected intravenously (i.v.) at a concentration of 2E7 PFU with one dose on the day of infusion. The anti-CD40, anti-PD1, anti-B7.1 and anti-B7.2 (anti-B7.1/2), and isotype control (IgG) antibodies were administered in doses of 100 μg via intraperitoneal (i.p.) 3 times every other day beginning on the day of infusion or intravenously (i.v.) once on the day of infusion. The peptides used were the human glycoprotein 100 (hgp100) peptide or the influenza nucleoprotein (Flu.NP) peptide which were injected subcutaneously (s.c.) or intravenously (i.v.) at a concentration of 100 μg as a single dose on the day of infusion.

### Tumor Size Assessment

Tumor treatment and measurement were conducted by independent investigators in a double-blinded manner. Two perpendicular diameters were measured using a caliper and the tumor size was calculated by the product of the two diameters.

### Probability of Survival Assessment

Probability of survival was assessed by measuring the percentage of surviving mice (n=5) on each day post-ACT in each treatment group.

### Statistics

Comparisons between groups were made using Wilcox rank-sum tests. In the Figures, NS, *, **, and *** indicate: not significant, p<0.05, <0.01, and <0.001 respectively.

### Example 1

This example demonstrates the use of CD39^{hi} Pmel T cells as a model for terminally exhausted T cells.

To establish a model for terminally exhausted T cells, mice from a line with a melanocyte protein (Pmel) mutation were injected with 100 μg/ml of human glycoprotein 100 (hgp100) peptide. After 5 days, those mice were injected with anti-CD3/anti-CD28 antibodies. On day 11, B16⁺ mice were treated with ACT by a transfer of the Pmel T cells expressing CD39 at low (CD39^{lo}), medium (CD39^{med}) or high (CD39^{hi}) levels. Isotype controls were used to distinguish CD39 expression. Within the CD39 gate, the bottom tertile expression was designated as CD39^{lo}, the middle tertile was designated as CD39^{med}, and the upper tertile was designated as CD39^{hi}.

Then B16⁺ tumor-bearing mice, which are a model for melanoma, were infused with transferred CD39^{med} or high CD39^{hi} T cells extracted from the Pmel mice and subsequently given three injections of interleukin 2 (IL-2) and monitored for tumor growth (FIG. 2A). CD39^{low}, CD39^{med}, and CD39^{hi} exhausted T cells were tested for phenotypic T cell failure after two stimulations in vitro and administration of a moderate cell dose of the stimulated cells, 7.5e5-1e6, to the B16⁺ tumor-bearing mice. Pmel⁺ T cells were isolated and categorized by CD39 expression level as low (CD39^{lo}), medium (CD39^{med}), or high (CD39^{hi}), as shown in FIG. 2B. CD39^{lo} cells had lower levels of TIM3 and PD1 expression than CD39^{hi} cells. CD39^{lo} and CD39^{hi} T cells were co-cultured with B16_{hgp100} Mel cells and 4-1BB activation was measured as an indicator of cell exhaustion. CD39^{lo} T cells had higher 4-1BB activation than CD39^{hi} T cells, and thus were less exhausted (FIG. 2C).

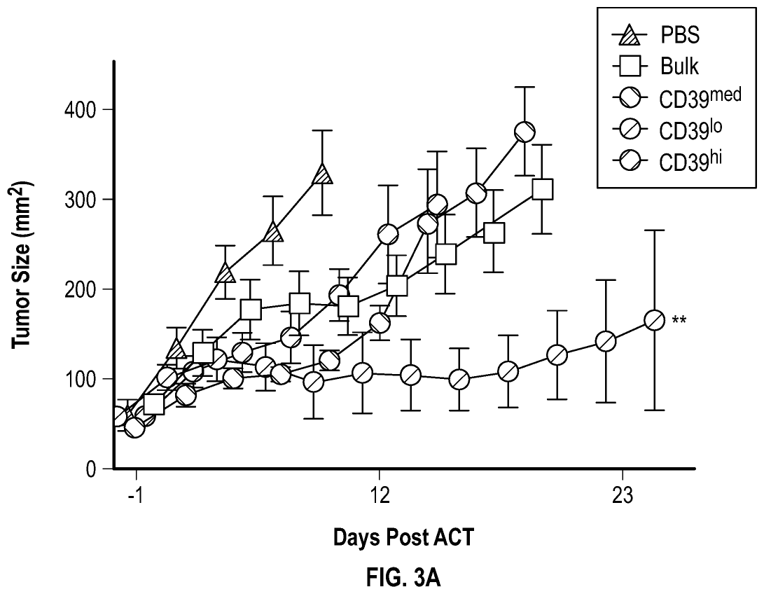

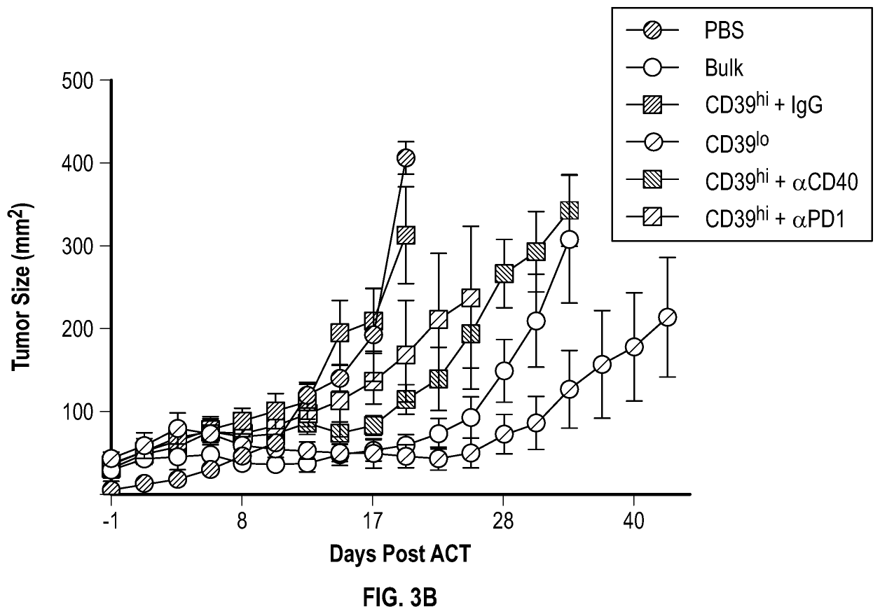

Tumor size was monitored for several days after ACT treatment. CD39^{med}, CD39^{hi}, PBS, and bulk control treatments did not control progression of B16_{hgp100} tumors. However, CD39^{lo} treatments significantly delayed tumor progression (FIG. 3A). CD39^{hi}+anti-PD1 antibody and CD39^{hi}+anti-CD40 antibody treatments controlled tumor progression better than CD39^{hi} T cells alone, but not as well as less-exhausted CD39^{lo} cells alone (FIG. 3B). These results indicated that T cells from Pmel mice can serve as an effective model for exhausted T cells.

### Example 2

This example demonstrates that hgp100 neoantigen vaccine can rescue CD39^{hi} exhausted antitumor T cells.

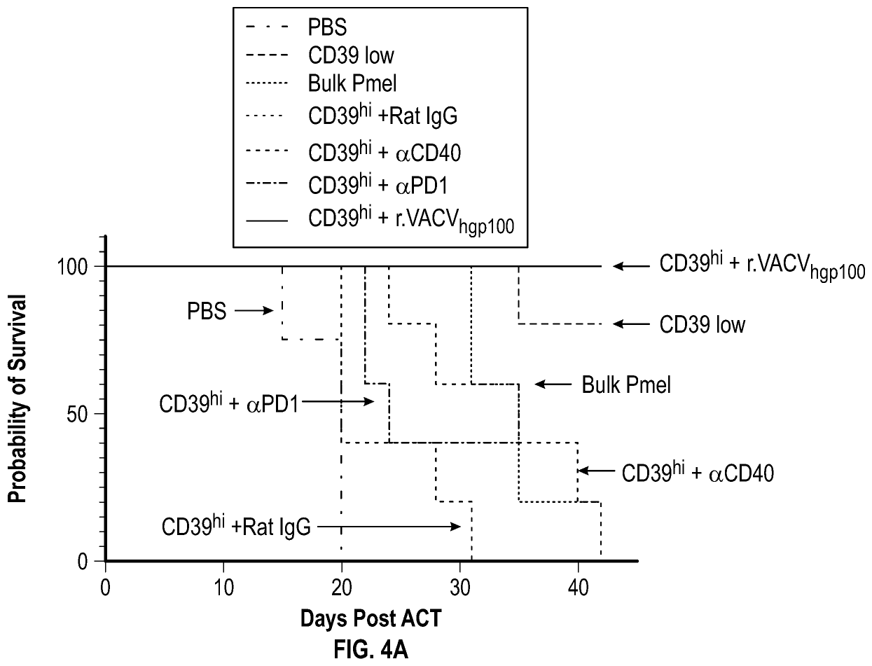

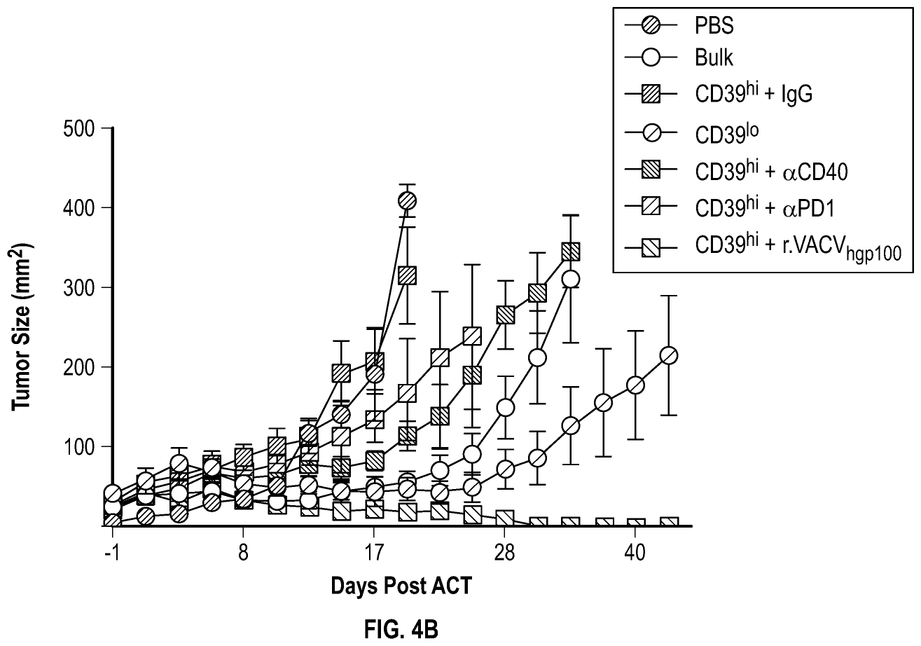

The ACT treatment described in Example 1 was repeated with an additional treatment group of CD39^{hi}+hgp100 neoantigen vaccination (r.VACV_{hgp100}). The following dosages were administered to the applicable treatment groups shown in FIGS. 4A and 4B: VACV: 2E7 PFU 1 dose; anti-CD40 antibody 100 μg (i.p.) 3 doses; anti-PD1 antibody 100 μg (i.p.) 3 doses. The CD39^{hi}+r.VACV_{hgp100} treatment group had 100% probability of survival after 40 days, while CD39^{lo} had an 80% chance of survival, and the remaining treatment groups all had a 20% probability or less of survival (FIG. 4A). The CD39^{hi}+r.VACV_{hgp100} treatment also controlled tumor progression more effectively than all other treatment groups and was the only treatment to shrink the tumors (FIG. 4B). The results showed that tumor treatment effects in bulk could largely be captured by less-exhausted CD39^{lo} T cells. The results also showed that VACV.hgp100 i.v. rescued CD39^{hi} Pmel antitumor T cells. The mice in these experiments had small tumors.

### Example 3

This example demonstrates the testing of different neoantigen vaccine modalities with ACT of CD39^{hi} exhausted antitumor T cells.

To further test neoantigen vaccine modalities with ACT, more experiments were performed on mice with large tumors, approximately 200 mm. B16⁺ mice were infused with either CD39^{hi} (more exhausted) or CD39^{lo} (less exhausted) T cells and, in combination, were given either (i) a single intravenous (i.v.) dose of VACV_{100hgp} on the day of cell infusion, (ii) a single i.v. dose of ADV_{hgp100} on the day of cell infusion, (iii) three intraperitoneal (i.p.) doses of 100 μg of anti-CD40 antibody every other day beginning on the day of cell infusion, or (iv) a single subcutaneous (s.c.) dose of 100 μg hgp100 peptide on the day of infusion with three intraperitoneal (i.p.) doses of 100 μg of anti-CD40 antibody every other day beginning on the day of cell infusion.

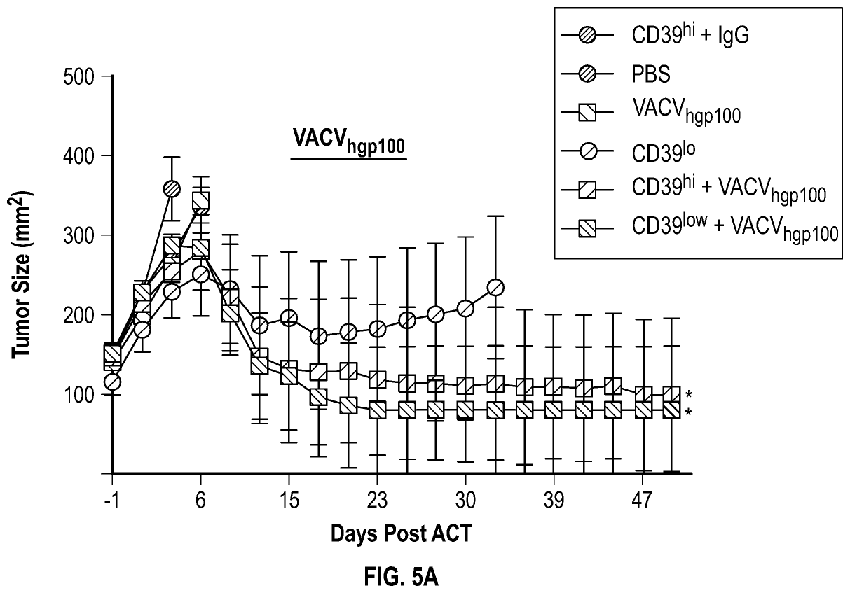

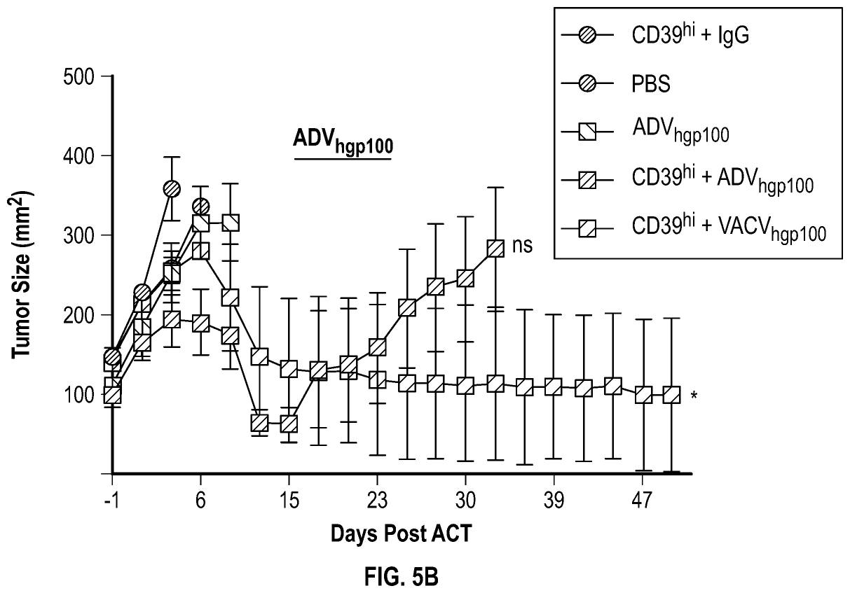

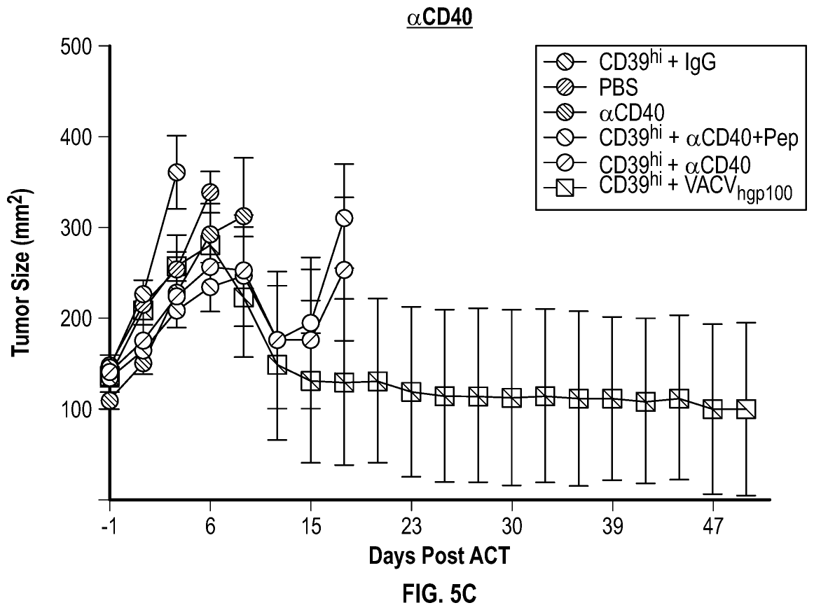

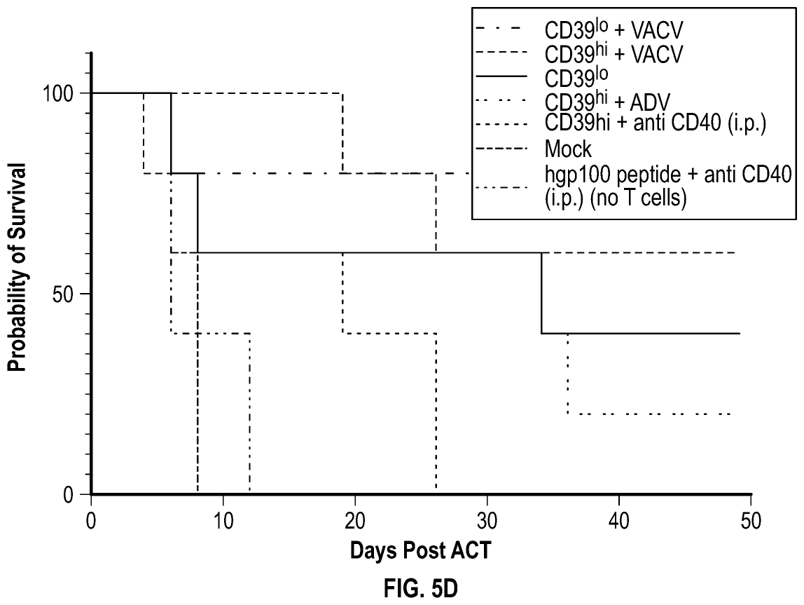

The CD39^{hi}+VACV_{100hgp} treatment was able to significantly delay tumor progression relative to control CD39^{hi} treatment alone (FIG. 5A). The CD39^{hi}+ADV_{hgp100} treatment (FIG. 5B) and the various anti-CD40 antibody treatments (FIG. 5C) both delayed tumor progression somewhat, but both less effectively than the CD39^{hi}+VACV_{100hgp} treatment. The probability of survival at 50 days post-ACT was highest for CD39^{lo}+VACV_{100hgp} (80%) and CD39^{hi}+VACV_{100hgp} (60%) treatments, but the CD39lo+ADV_{hgp100} (40%) and CD39^{hi}+ADV_{hgp100} (20%) treatments also increased survival relative to all other treatments (0%) (FIG. 5D). CD39lo less-exhausted T cells alone served as a control. These results indicated that the neoantigen-specific vaccine was able to rescue the ability of exhausted T cells to slow tumor progression and increase the probability of survival.

### Example 4

This example demonstrates that a neoantigen vaccine rescues different subsets of exhausted antitumor T cells.

Vaccine-mediated rescue is not limited to just CD39^{hi} exhausted antitumor T cells.

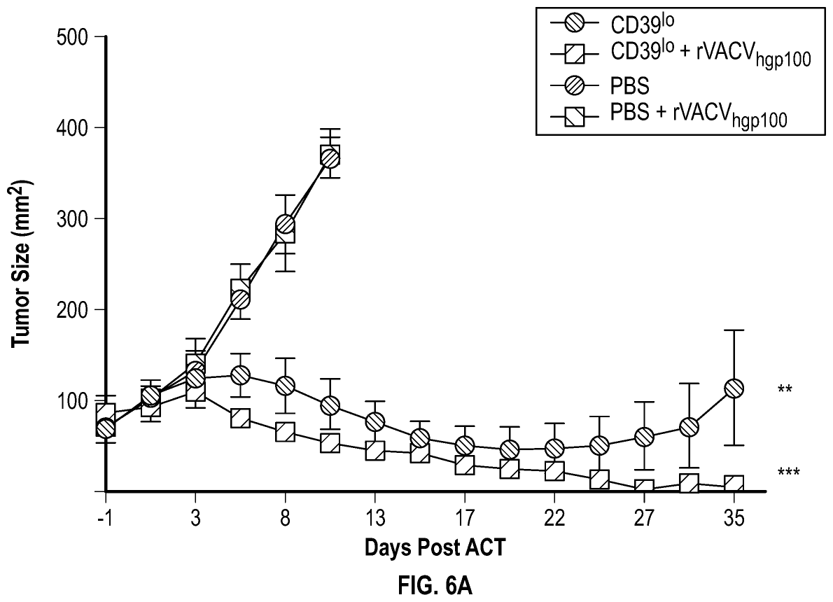

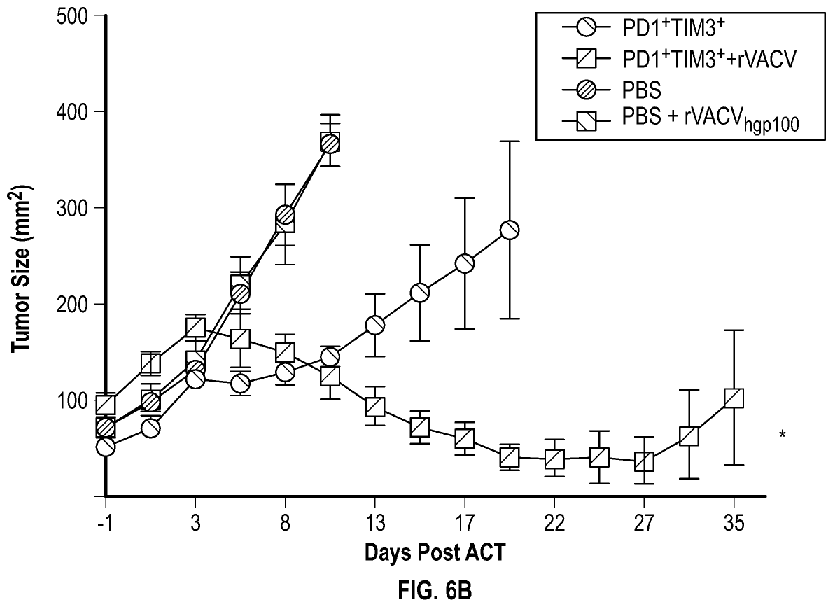

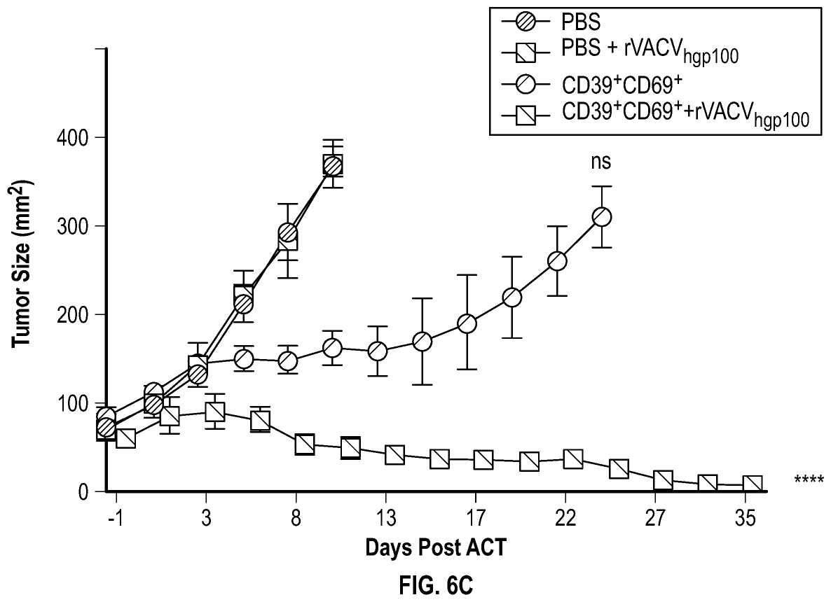

The tumor-bearing mice described in Example 1 were treated with PD1⁺TIM3⁺ cells (alone or in combination with rVACV_{100hgp}) or PBS (alone or with rVACV_{100hgp}). PD1⁺TIM3⁺ terminally exhausted neoantigen-specific T cells were also rescued with a neoantigen vaccine (FIG. 6B).

The tumor-bearing mice described in Example 1 were treated with CD39⁺ CD69⁺ cells (alone or in combination with rVACV_{100hgp}) or PBS (alone or with rVACV_{100hgp}). Neoantigen vaccine also rescued tumor progression in CD39⁺ CD69⁺ terminally exhausted neoantigen-specific T cells (FIG. 6C).

The tumor-bearing mice described in Example 1 were treated with CD39lo cells (alone or in combination with rVACV_{100hgp}) or PBS (alone or with rVACV_{100hgp}). Neoantigen vaccine also rescued CD39^{lo} less-exhausted antitumor cells, although CD39^{lo} treatment alone also inhibited tumor progression (FIG. 6A).

### Example 5

This example demonstrates that a relevant neoepitope is required for exhausted T cell rescue by vaccine during ACT.

Irrelevant HLA-A2 restricted hgp100 epitope (hgp100₂₀₉) and relevant hgp100 epitope (hgp100₂₅) were tested to determine the neoepitope requirements for exhausted T cell rescue. The tumor-bearing mice described in Example 1 were treated with PBS alone or in combination with hgp100₂₀₉ or hgp100₂₅ or were treated with CD39^{lo} T cells alone or in combination with hgp100₂₀₉ or hgp100₂₅.

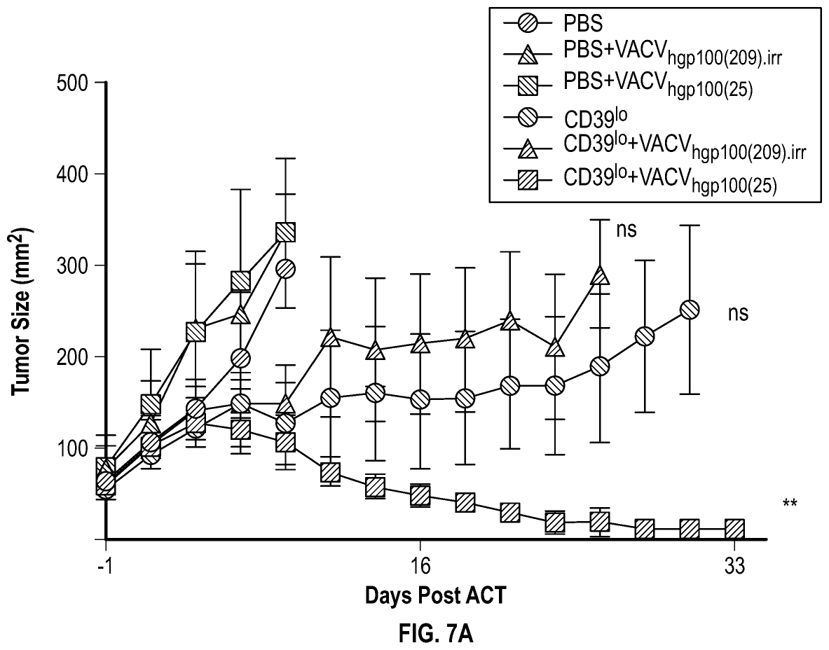

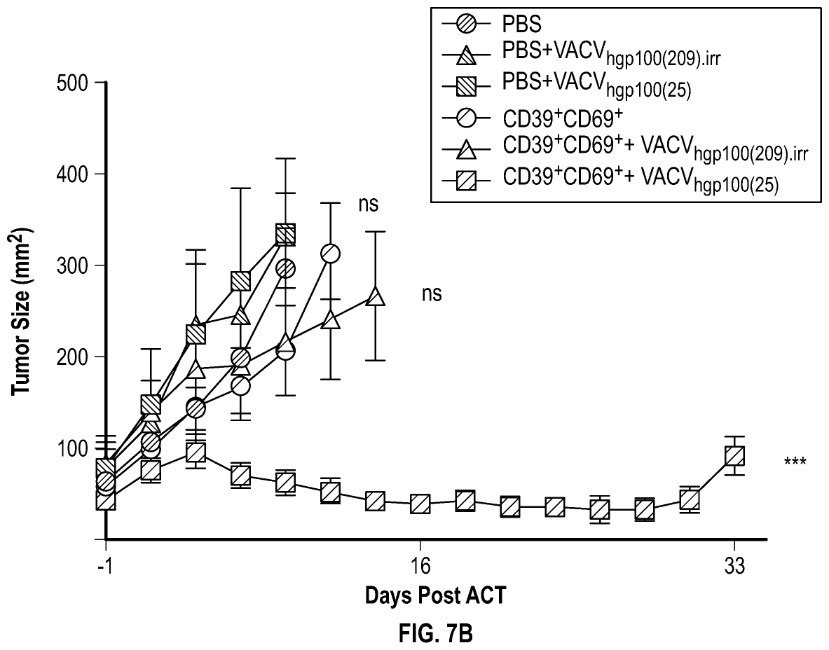

CD39^{lo} less exhausted T cells were only able to inhibit tumor progression in combination with relevant VACV_{hgp100(25)} (FIG. 7A). CD39^{lo} T cell treatment alone was unable to significantly inhibit tumor progression in this experiment (FIG. 7A). CD39⁺ CD69⁺ exhausted neoantigen-specific T cells were also only able to inhibit tumor progression in combination with relevant VACV_{hgp100(25)} (FIG. 7B). These results demonstrated that relevant neoepitopes are required in the neoantigen vaccine for rescue of exhausted T cells anti-tumor activity.

### Example 6

This example demonstrates that a neoantigen vaccine increases the frequency of CD8⁺ exhausted T cells post-ACT.

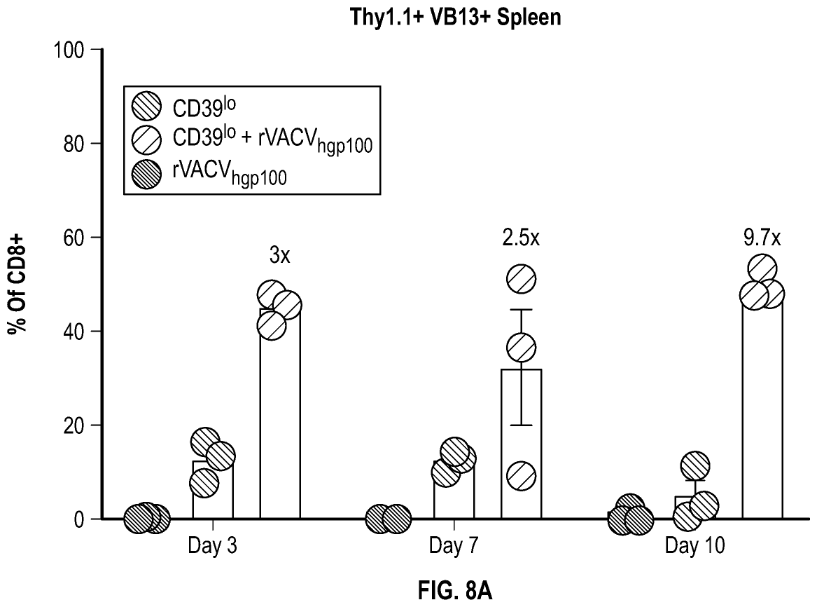

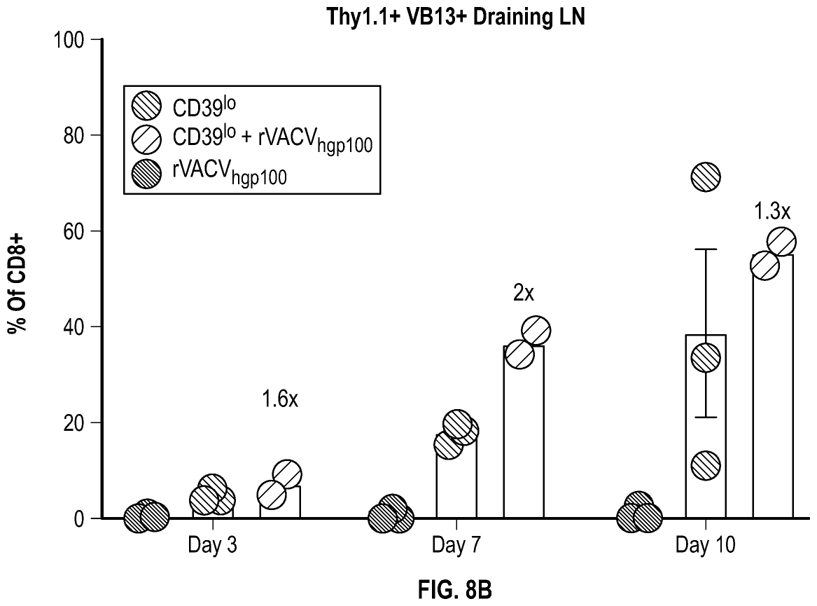

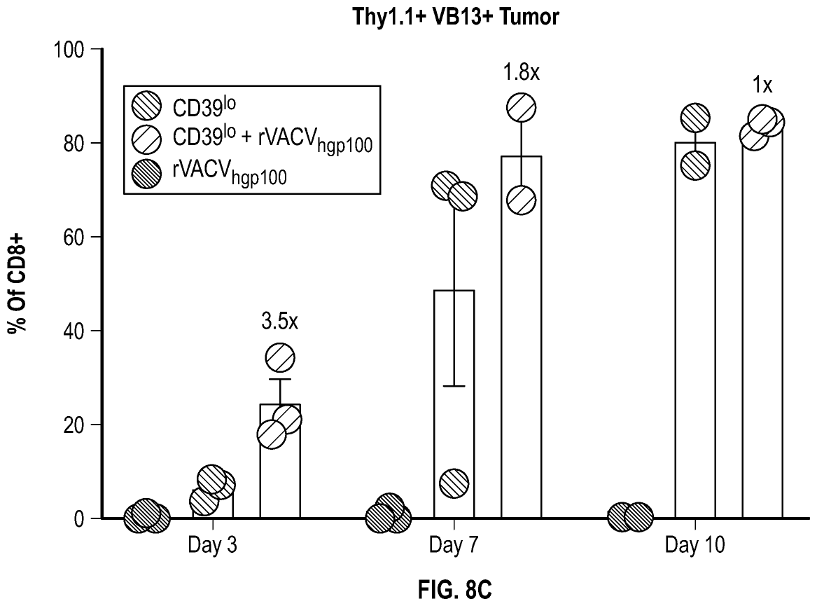

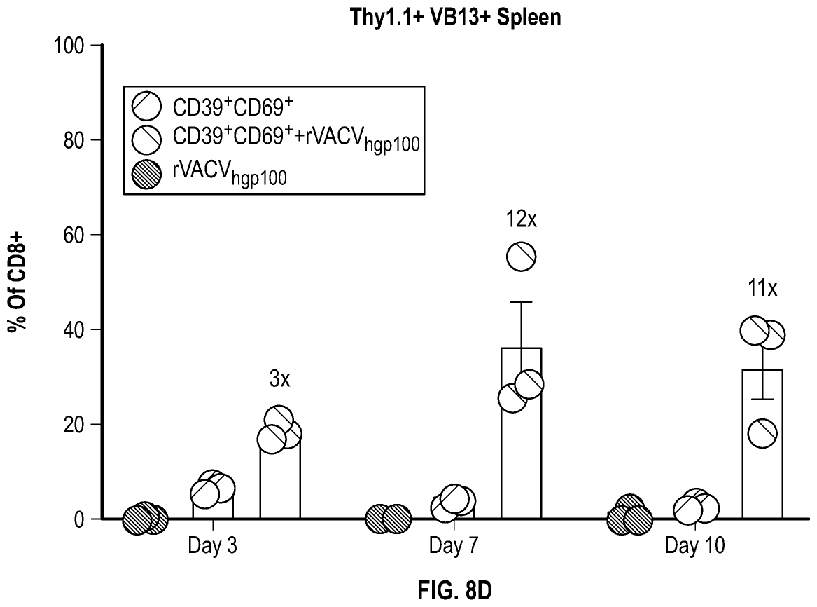

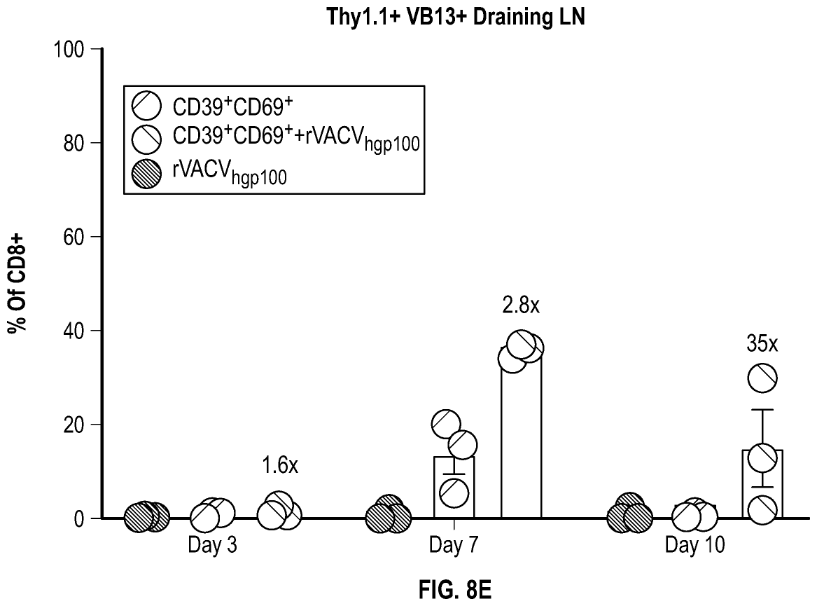

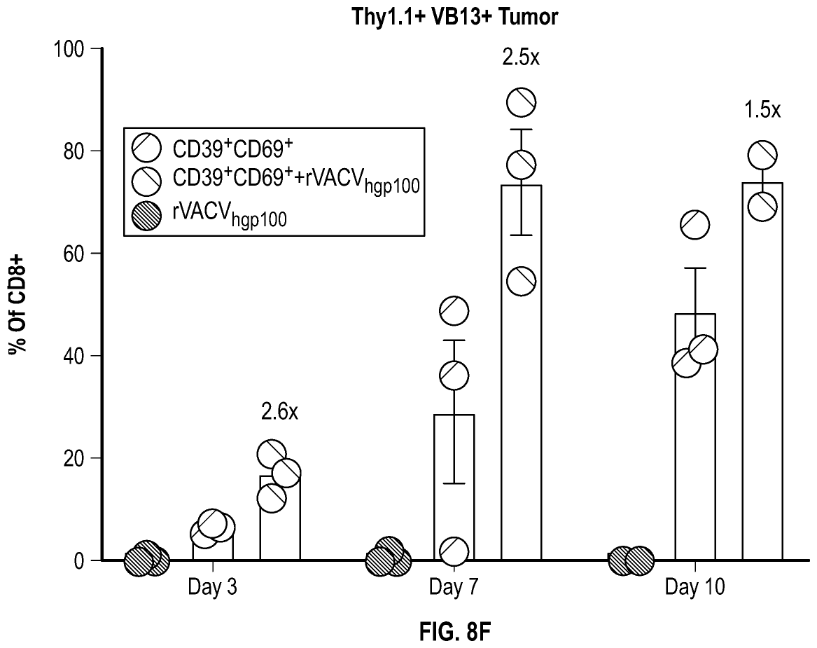

The tumor-bearing mice described in Example 1 were treated with CD39^{lo} T cells alone, rVACV_{hgp100} alone, or a combination of CD39^{lo} T cells and rVACV_{hgp100} (FIG. 8A-8C) or were treated with CD39⁺ CD69⁺ T cells alone, rVACV_{hgp100} alone, or a combination of CD39⁺ CD69⁺ T cells and rVACV_{hgp100} (FIG. 8D-8F).

To further investigate the effects of neoantigen vaccines on different subgroups of exhausted antitumor T cells, the percentage of Thy1.1⁺ Vβ13⁺ CD8⁺ transferred T cells was compared to total CD8+ T cells in the spleens, draining lymph nodes, and tumors of mice post-ACT.

rVACV_{hgp100} treatment following ACT infusion of CD39^{lo} less exhausted T cells increased the frequency of adoptively transferred T cells in the spleen (FIG. 8A), draining lymph node (FIG. 8B), and tumor (FIG. 8C) of B16⁺ mice on days 3, 7 and 10 post-ACT. There are no differences in transferred T cells on Day 10 post-ACT when using less-exhausted CD39^{lo} T cells. The difference is more apparent when using dysfunctional differentiated CD39^{hi} or CD39⁺ CD69⁺ T cells for ACT with the vaccine. rVACV_{hgp100} treatment following ACT infusion of CD39⁺69⁺ highly exhausted T cells also increased the frequency of adoptively transferred T cells in the spleen (FIG. 8D), draining lymph node (FIG. 8E), and tumor (FIG. 8F) of B16⁺ mice on days 3, 7 and 10 post-ACT. The fold change in transferred CD8⁺ cells post-ACT was significantly higher in the terminally exhausted T cells than the less exhausted T cells.

### Example 7

This example demonstrates that a neoantigen vaccine decreases the frequency of PD1⁺TIM3⁺ terminally exhausted T cells post-ACT.

The tumor-bearing mice described in Example 1 were treated with CD39^{lo} T cells alone or in combination with rVACV_{hgp100} or were treated with CD39⁺ CD69⁺ T cells alone or in combination with rVACV_{hgp100}.

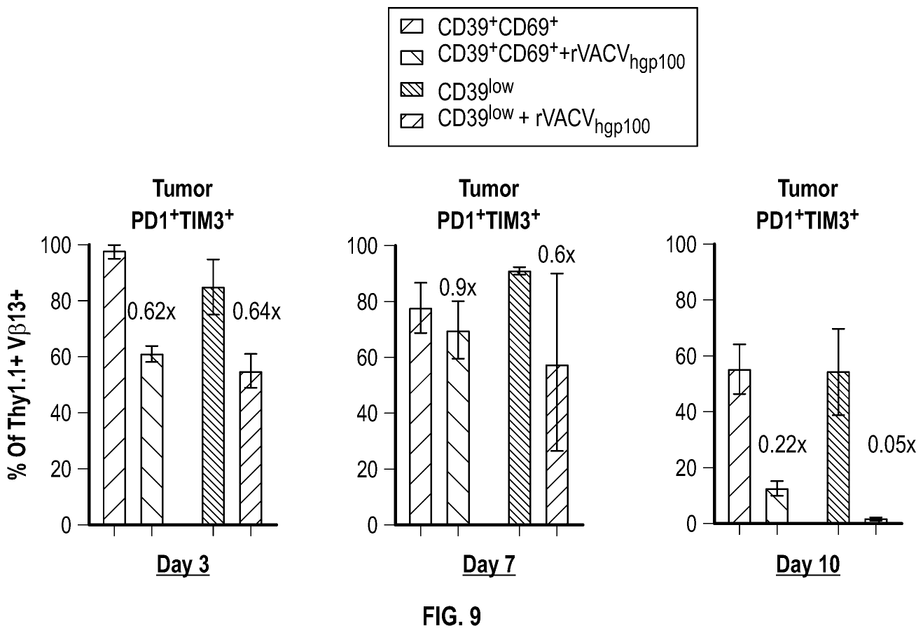

To further investigate the effects of neoantigen vaccines on exhausted antitumor T cells, the percentage of Thy1.1⁺ Vβ13⁺ neoantigen-specific transferred T cells was examined in the tumors of mice post-ACT. rVACV_{hgp100} treatment significantly decreased the frequency of PD1⁺TIM3⁺ adoptively transferred Thy1.1⁺ Vβ13⁺ hgp100 neoantigen-specific CD39^{lo} and CD39⁺CD69⁺ T cells as a percentage of the total live CD8⁺ T cells isolated from the tumor of mice on days 3 and 10 post-ACT (FIG. 9). These results suggested that neoantigen vaccination combined with ACT decreases the frequency of transferred exhausted T cells in the tumor post-ACT.

### Example 8

This example demonstrates that anti-CD40 antibody and neoepitope intravenous vaccination delays tumor progression during ACT with exhausted T cells.

To further investigate how the vaccine administration route impacts the success of vaccine-mediated rescue of exhausted T cells in ACT, additional experiments were performed. Previous experiments using intraperitoneal anti-CD40 antibody combined with peptide vaccination had a modest effect on ACT of terminally exhausted CD39⁺CD69⁺ T cells. So intravenous anti-CD40⁺ antibody and peptide was tested.

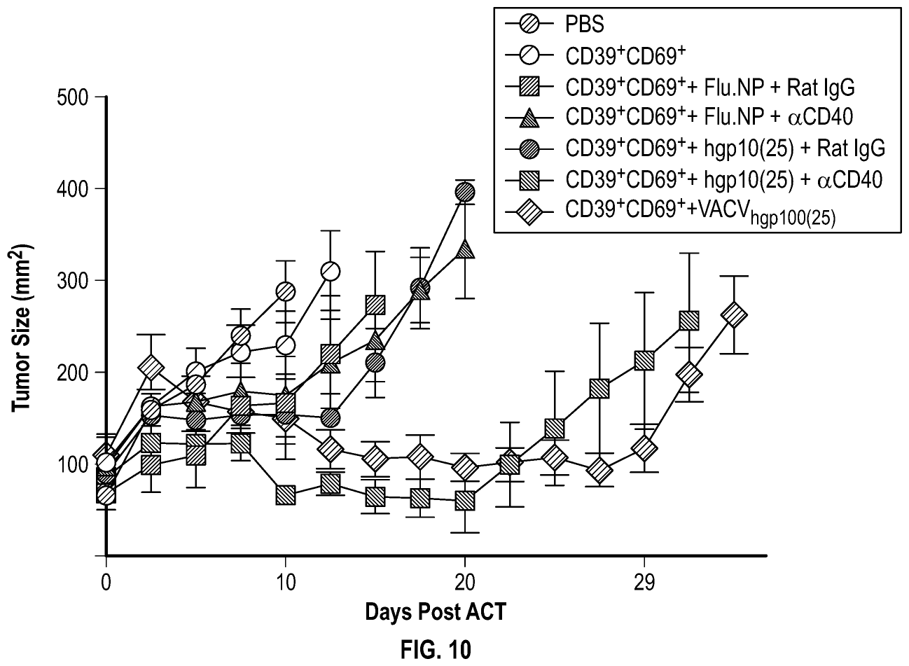

The results showed that vaccination delayed the effect on ACT of terminally exhausted CD39⁺CD69⁺ T cells (FIG. 10). The tumor-bearing mice described in Example 1 underwent ACT with CD39⁺CD69⁺ hgp100 neoantigen-specific Pmel T cells alone, or those T cells co-administered with vaccinia virus vaccine encoding hgp100(25-33), or relevant neoepitope (hgp100(25)) with or without co-administration of intravenous anti-CD40 or isotype control (Rat IgG), or irrelevant peptide (Flu.NP) with or without co-administration of intravenous anti-CD40 or Rat IgG. The inability of the irrelevant peptide to rescue exhausted T cell ACT further suggests that relevant neoepitope is a determinant of vaccine-mediated rescue of terminally differentiated dysfunctional T cell-based ACT (See Flu NP vs. hgp100). Additionally, this suggests that route of peptide or antibody administration, intravenous as opposed to intraperitoneal, may play a role in the success of vaccine-mediated rescue of exhausted T cell ACT. The similar effects that the peptide and anti-CD40 antibody had as the vaccinia virus vaccine had on tumor size suggests that non-vaccinia virus vaccines can also mediate ACT-rescue due to terminally exhausted CD39⁺CD69⁺ T cells.

### Example 9

This example demonstrates that dendritic cells loaded with neoepitope, administered as a vaccine along with ACT, delays tumor progression.

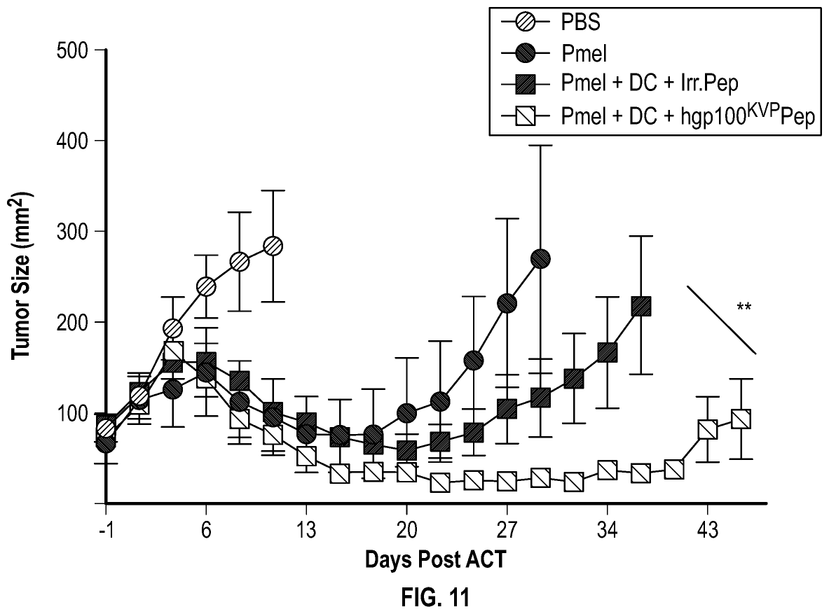

To investigate non-vaccinia virus vaccines, bone marrow derived dendritic cells (BMDCs) were tested. Tumor progression was measured in the tumor-bearing mice described in Example 1 that were treated with bulk, unsorted, transgenic hgp100 neoantigen-specific Pmel T cells alone (Pmel), or Pmel T cells co-administered with BMDCs. The BMDCs had been peptide pulsed for 4 hours with irrelevant influenza viral peptide (Pmel+DC+Irr.Pep) or with relevant neoepitope (Pmel+DC+hgp100^{KVP} Pep). Untreated mice (PBS) served is a control. The treatment using the BMDCs pulsed with the relevant neoepitope delayed tumor progression significantly more (P<0.01) than hgp100 neoantigen-specific Pmel T cells alone or co-administered with BMDCs pulsed with irrelevant peptide (FIG. 11). These data suggested that DC-based vaccines can also mediate tumor regression through differentiated T cell ACT.

### Example 10

This example demonstrates that a neoantigen vaccine with exhausted T cell ACT delays tumor progression in a colon tumor model.

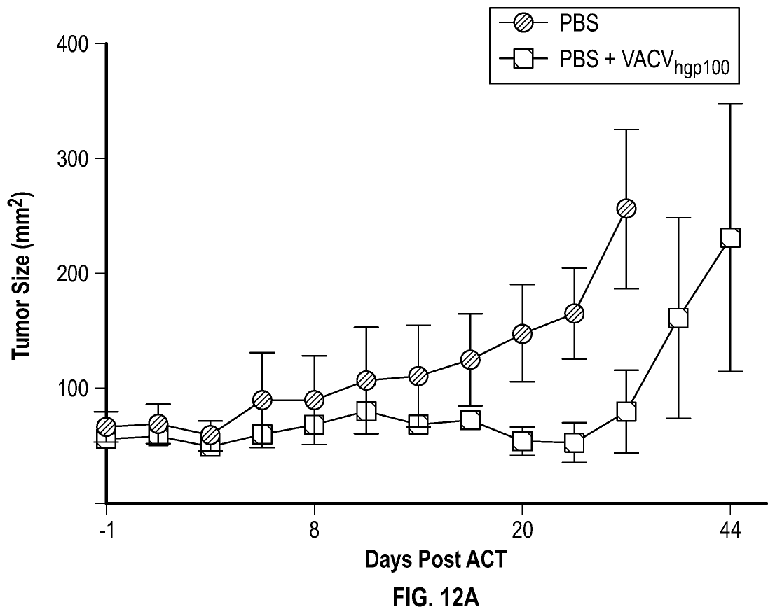

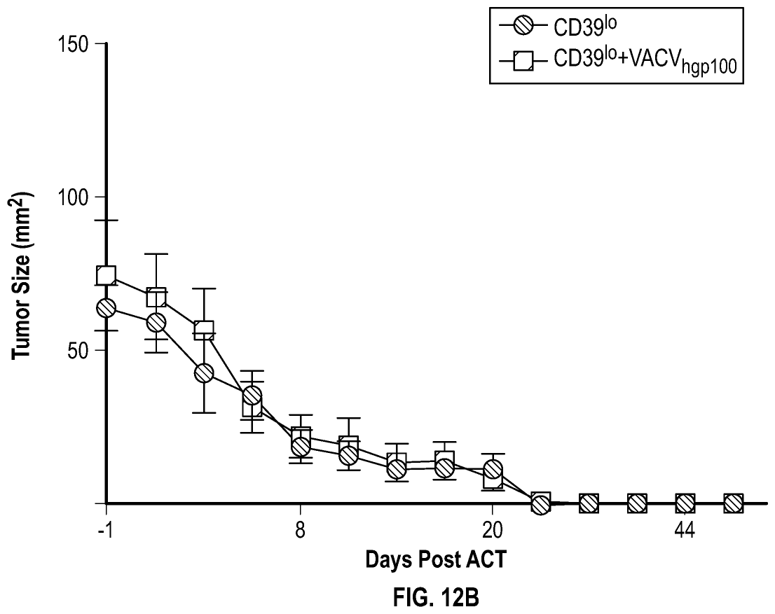

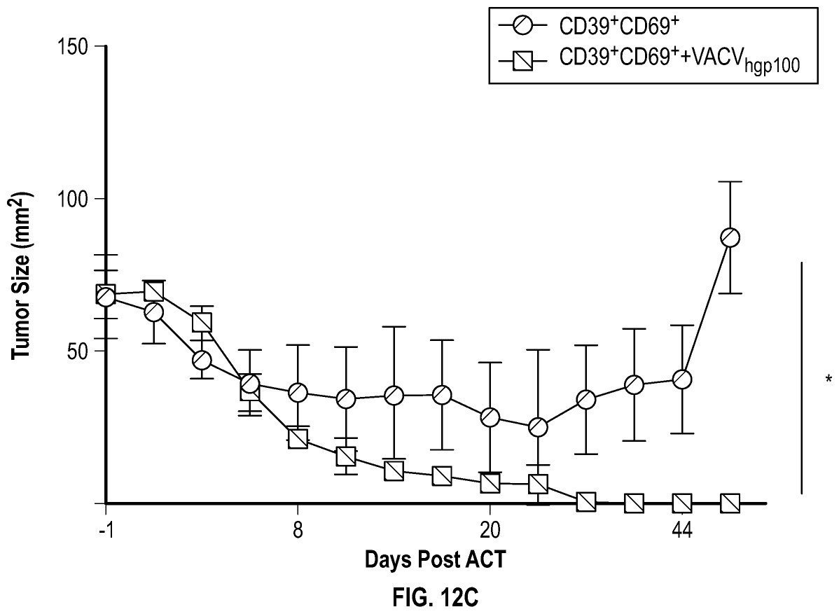

In order to test the efficacy of neoantigen vaccine with exhausted T cell ACT in a non-melanoma tumor model, experiments were performed testing tumor progression in MC38 colon cancer tumors expressing the hgp100KVP neoepitope. Vaccinia virus expressing the same hgp100KVP neoepitope was used as a concurrent neoantigen vaccine. When administered alone, the neoantigen vaccine had no significant effect on tumor progression compared with untreated (PBS) (FIG. 12A). MC38 tumor progression was also measured after ACT with less exhausted CD39^{lo} Pmel transgenic T cells alone or with co-administration of neoantigen vaccine, both of which mediated long term tumor control (FIG. 12B). MC38 tumor progression was tested after ACT with co-administration of neoantigen vaccine with terminally exhausted CD39⁺CD69⁺ Pmel transgenic T cells, which decreased tumor progression significantly more (P<0.01) than CD39⁺CD69⁺ Pmel transgenic T cells alone. CD39⁺CD69⁺ Pmel transgenic T cells alone were unable to mediate tumor control (FIG. 12C). These data suggested that ACT with neoantigen vaccine mediates durable tumor regression of large tumors in both melanoma and non-melanoma tumor models.

### Example 11

This example demonstrates that antigen presentation by host cells is required for exhausted T cell rescue by vaccine.

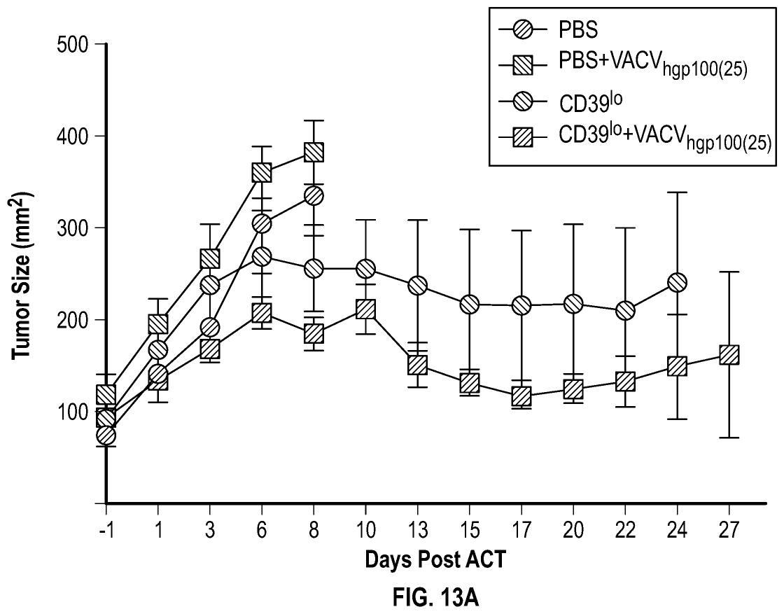

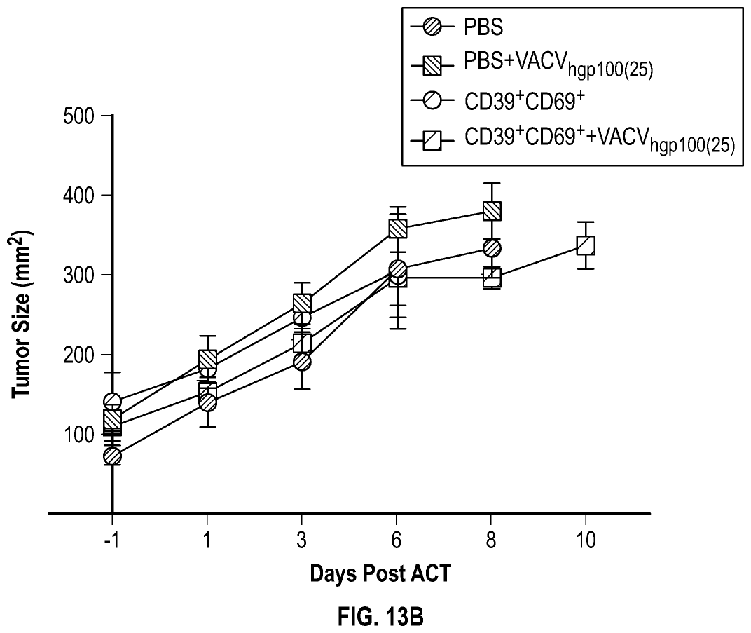

To investigate the role of antigen presentation by host cells for exhausted T cell rescue by neoantigen-specific vaccine, experiments were performed in β2 microglobulin knockout (β2M KO) mice. Tumor progression was measured in β2M KO mice post-ACT with CD39^{lo} Pmel transgenic T cells alone, T cells co-administered with vaccinia virus vaccine encoding hgp100(25-33), the vaccinia virus alone, or untreated (PBS). The CD39^{lo} less-exhausted neoantigen-specific T cells were not impacted during ACT in the β2M KO mouse body (FIG. 13A). However, when a similar experiment was performed using terminally exhausted CD39⁺CD69⁺ Pmel transgenic T cells, they could not be rescued with neoantigen vaccine (FIG. 13B). This suggests that antigen presentation from host cells might be required for rescue of exhausted T cell ACT.

### Example 12

This example demonstrates that exhausted T cell rescue by vaccine is impacted by B7.1/B7.2 blockade.

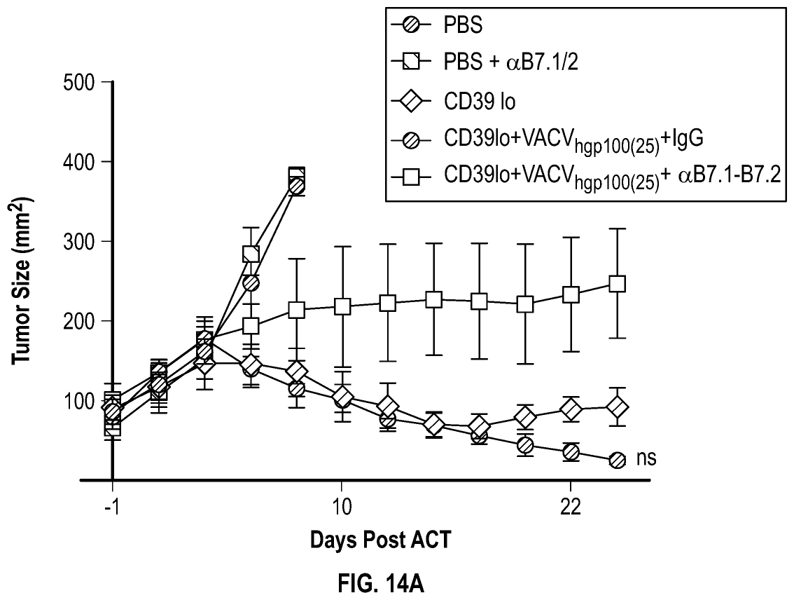

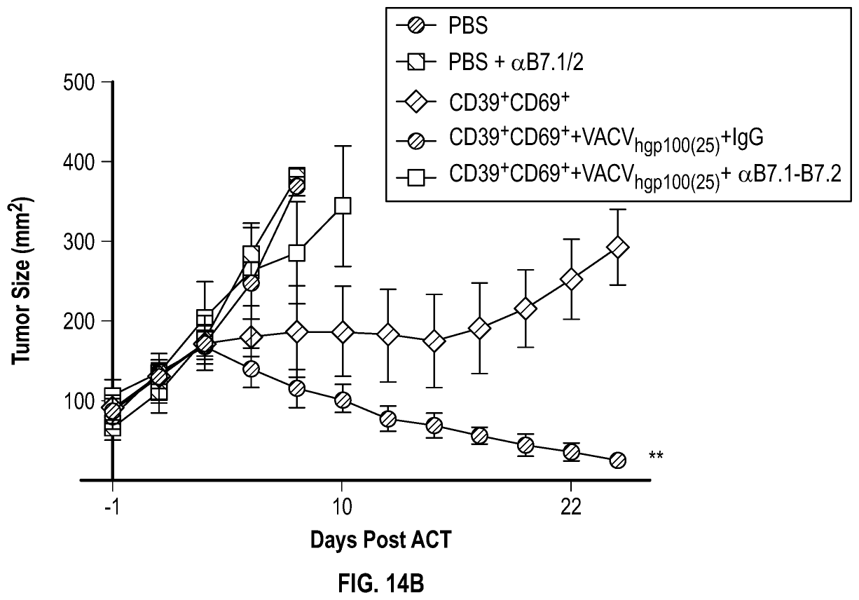

To further investigate the mechanisms of neoantigen vaccine rescue of exhausted T cells, the roles of B7.1 (CD80) and B7.2 (CD86) were tested. Tumor progression was tested after ACT with less exhausted CD39^{lo} Pmel transgenic T cells alone or with co-administration of neoantigen vaccine with or without blockade of B7.1 and B7.2, using anti-B7.1 and anti-B7.2 (anti-B7.1/2) antibodies (100 μg) or isotype control (100 μg), or untreated (PBS) or untreated with B7.1/B7.2 blockade (PBS+anti-B7.1/2) (FIG. 14A). The same experiment was performed using terminally exhausted CD39⁺CD69⁺ Pmel transgenic T cells (FIG. 14B). The vaccinia virus encoding neoantigen mediated rescue of CD39⁺CD69⁺ terminally exhausted neoantigen-specific T cells was significantly (P<0.01) impacted by blockade of B7.1/B7.2, suggesting that co-stimulation of host antigen-presenting cells (APCs) might be involved in vaccine-mediated rescue of terminally differentiated exhausted T cell ACT. The blockade of B7.1/B7.2 had a lower impact on CD39^{lo} less-exhausted neoantigen-specific T cell ACT.

### Example 13

This example demonstrates retrospective evidence of exhausted T cell rescue by vaccine-based treatment of a human patient with TIL ACT.

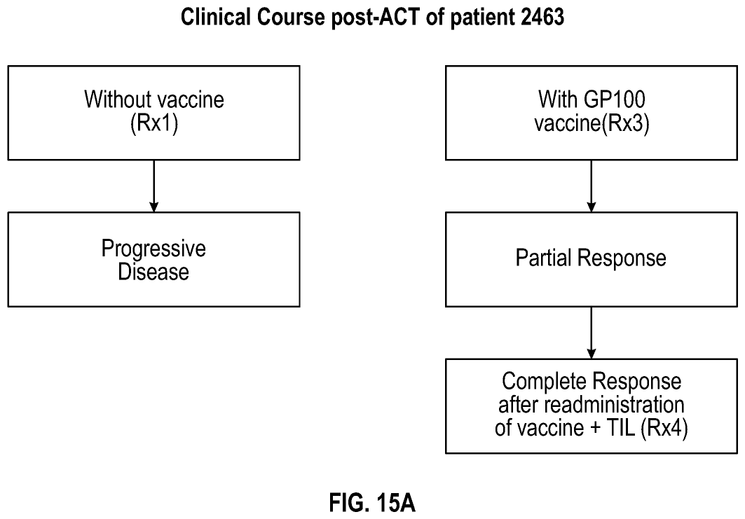

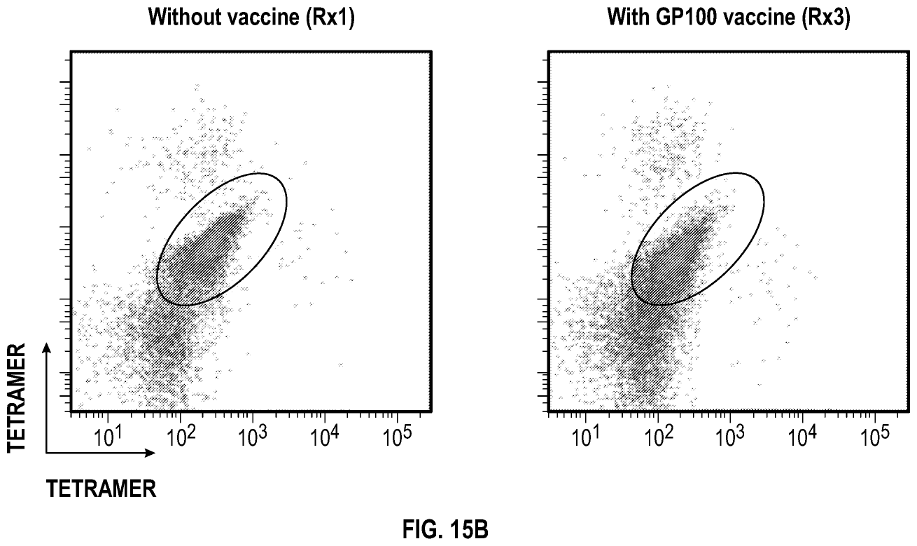

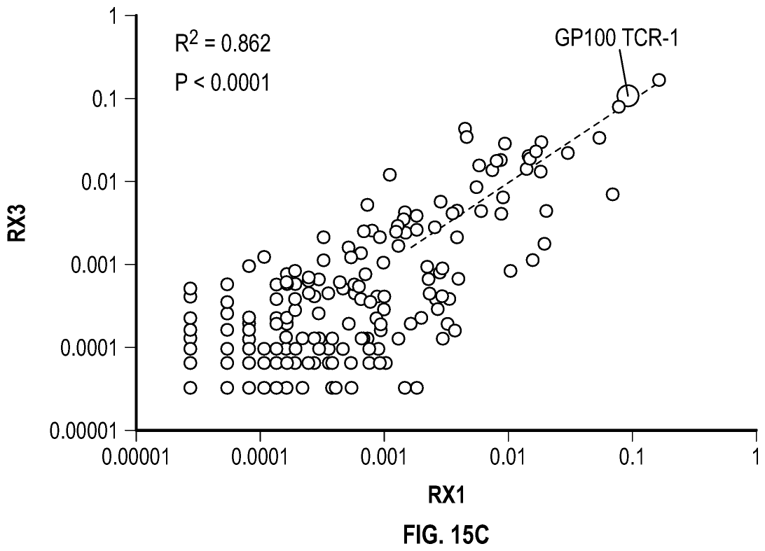

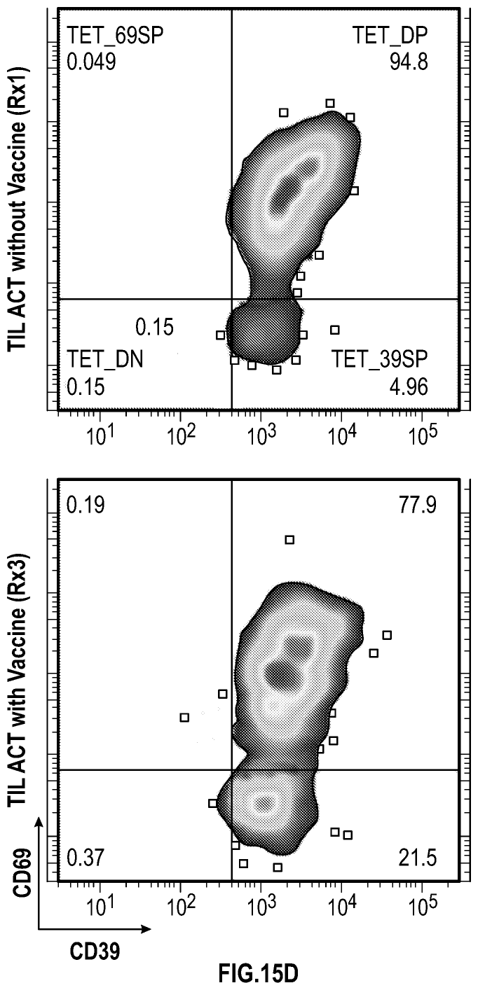

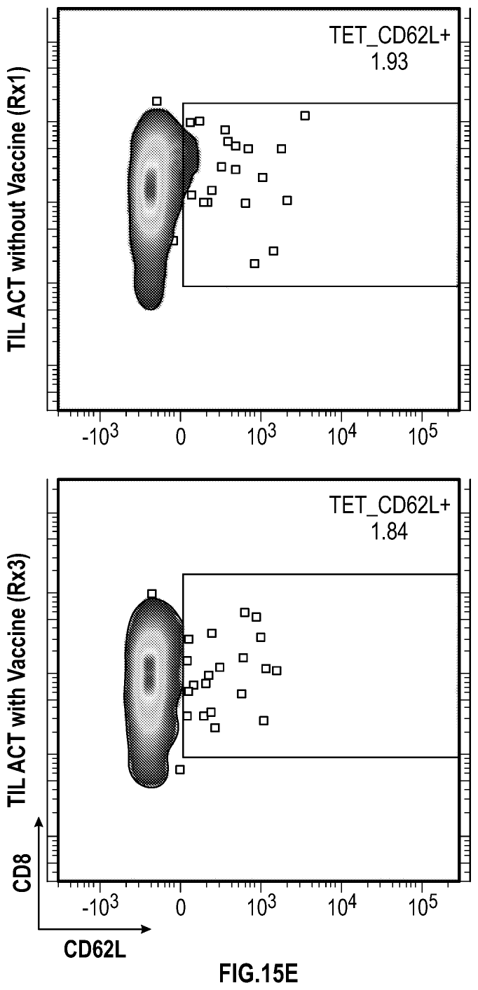

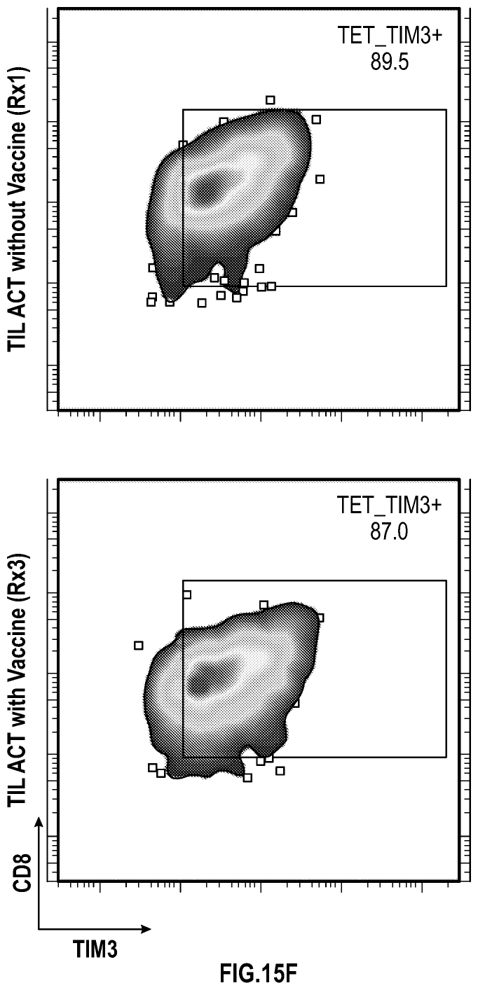

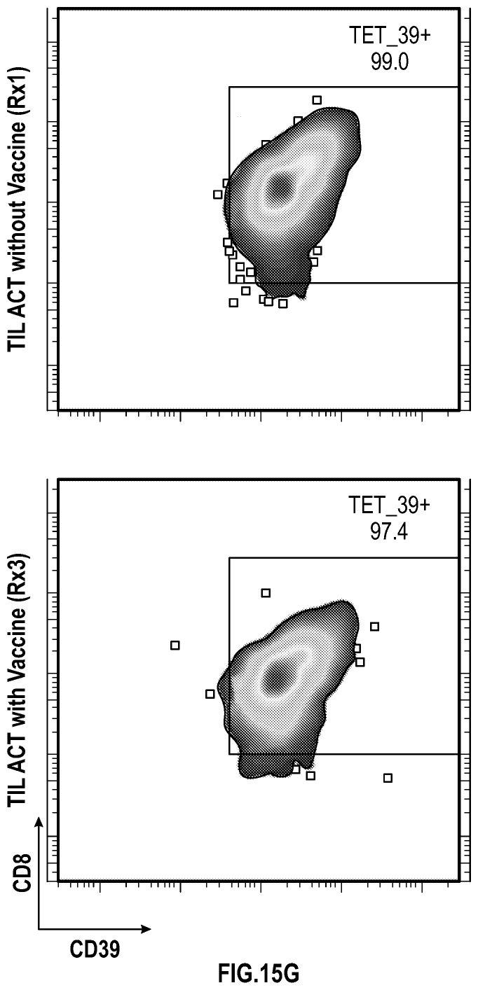

Further evidence for the efficacy of exhausted T cell rescue by neoantigen vaccine was provided by the clinical course of melanoma Patient 2463 treated with tumor infiltrating lymphocyte (TIL) therapy with and without vaccine. Patient 2463 was first treated with TIL only (intravenous TIL (Rx1) and intra-arterial TIL (Rx2)). After progressive disease, the patient was retreated with TIL+fowlpox vaccine encoding the tumor antigen GP100. Rx1 and Rx3 refer to first and third treatments using TIL infusion products, respectively. The patient had intra-arterial TIL as an infusion product 2 (Rx2) with no response. Rx2 was excluded from this analysis since it was administered via intra-arterial TIL administration. This clinical course was published in Smith et al, J. Immunother., 32(8): 870-874 (2009) (FIG. 15A).

TIL infusion products administered in the first intravenous administration (Rx1) and second intravenous administration concurrently with the GP100 fowlpox vaccine (Rx3) after failure of the first two TIL therapies had comparable GP100-specific TIL by tetramer staining (FIG. 15B). The numbers indicated the HLA-A0201-restricted GP100 tetramer frequencies in Rx1 and Rx3 as a percentage of CD8+ TIL. These data suggested that the TIL infusion administered with the vaccine that mediated response did not have more antitumor TIL compared to Rx1, which did not mediate clinical response.

The TCR clonal frequencies were highly correlated between Rx1 and Rx3 (FIG. 15C). The labeled immunodominant GP100 TCR-1 identified from patient 2463 TIL showed that the clonal repertoire was the same between Rx1 and Rx3, suggesting that the GP100 fowlpox vaccine acted on similarly frequent antitumor TILs in the infusion product.

Phenotypic states of antitumor GP100 TILs were comparable between Rx1 and Rx3. The phenotypes within GP100 tetramer-positive antitumor TILs between Rx1 and Rx3 based on the numbers indicated percentages within tetramer-positive TILs. Thus, the fowlpox GP100 vaccine co-administered with TIL likely acted on dysfunctional antitumor TIL, as shown by very low frequencies of CD39⁻CD69⁻ and higher frequency of CD39⁺CD69⁺ (FIG. 15D), and single markers CD62L⁺ (FIG. 15E), TIM3⁻ (FIG. 15F), or CD39⁻ (FIG. 15G) cells within tetramer⁺ TIL in infusion products. This clinical case suggested that neoantigen vaccine rescue of exhausted T cells will be effective for mediation of long term control in humans.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1-35. (canceled)

36. A method of treating or preventing cancer in a mammal, the method comprising:
(a) isolating T cells from a tumor sample from the mammal,
wherein the isolated T cells are one or both of exhausted and differentiated, and the isolated T cells have antigenic specificity for a tumor-specific antigen expressed by the tumor sample from the mammal,
wherein the tumor-specific antigen is a tumor-specific neoantigen or an antigen with a tumor-specific driver mutation; and
optionally expanding the numbers of isolated, tumor antigen-specific T cells; and
(b) administering to the mammal (i) the isolated T cells of (a) and (ii) a vaccine which specifically stimulates an immune response against the tumor-specific antigen for which the isolated T cells have antigenic specificity.

37. The method of claim 36, comprising expanding the numbers of isolated, tumor antigen-specific T cells.

38. A method of treating or preventing cancer in a mammal with a tumor, the method comprising:
(a) isolating T cells from a biological sample from the mammal with the tumor;
(b) introducing into the isolated T cells a nucleic acid comprising a nucleotide sequence encoding an exogenous receptor having antigenic specificity for a tumor-specific antigen expressed by the tumor of the mammal to produce T cells which express the exogenous receptor, wherein the tumor-specific antigen is a tumor-specific neoantigen or an antigen with a tumor-specific driver mutation; and
optionally expanding the numbers of T cells which express the exogenous receptor; and
(c) administering to the mammal (i) the T cells which express the exogenous receptor of (b) and (ii) a vaccine which specifically stimulates an immune response against the tumor-specific antigen for which the exogenous receptor has antigenic specificity.

39. The method of claim 38, comprising expanding the numbers of T cells which express the exogenous receptor.

40. The method of claim 38, wherein the T cells isolated from the biological sample are one or both of exhausted and differentiated.

41. The method of claim 38, wherein the biological sample is a sample of the tumor.

42. The method of claim 36, wherein the isolated T cells are TIL.

43. The method of claim 38, wherein the biological sample is a peripheral blood sample.

44. The method of claim 38, wherein the exogenous receptor is a T cell receptor (TCR).

45. The method of claim 38, wherein the exogenous receptor is a chimeric antigen receptor (CAR).

46. The method of claim 36, wherein (i) and (ii) are administered to the mammal simultaneously.

47. The method of claim 36, wherein (i) and (ii) are administered to the mammal together in the same composition.

48. The method of claim 36, wherein (i) and (ii) are administered to the mammal sequentially.

49. The method of claim 48, wherein (i) is administered to the mammal before (ii).

50. The method of claim 48, wherein (ii) is administered to the mammal before (i).

51. The method of claim 48, wherein (i) is administered to the mammal within 24 hours before (ii) is administered to the mammal.

52. The method of claim 48, wherein (i) is administered to the mammal within 24 hours after (ii) is administered to the mammal.

53. The method of claim 36, wherein the tumor-specific driver mutation is mutated ALK, mutated APC, mutated ATRX, mutated BRAF, mutated CDKN2A, mutated DDX3X, mutated DNMT3A, mutated EGFR, mutated ESR1, mutated EWSR1, mutated FGFR1, mutated FLI1, mutated HRAS, mutated IDH1, mutated IDH2, mutated KMT2C, mutated KRAS, mutated MYC, mutated NOTCH1, mutated NRAS, mutated PIK3CA, mutated PTCH1, mutated PTEN, mutated RB1, mutated RUNX1, mutated SETD2, mutated SMARCA4, mutated STK11, or mutated TP53.

54. The method of claim 36, wherein the vaccine is a cancer cell vaccine, a conjugate polysaccharide vaccine, a dendritic cell vaccine, a DNA vaccine, an inactivated vaccine, a live-attenuated vaccine, a nanoparticle vaccine, a peptide vaccine, a protein vaccine, a recombinant vaccine, an RNA vaccine, a subunit vaccine, or a viral vaccine.

55. The method of claim 36, wherein the isolated T cells express any one or more of the following markers of T cell exhaustion:
(a) RNA encoding any one or more of: 4-1BB⁺, CCL3⁺, CD28⁻, CD39⁺, CD62L⁻ (SELL⁻), CD69⁺, CTLA4⁺, CX3CR1⁺, CXCL13⁺, CXCR6⁺, GZMA⁺, GZMB⁺, GZMK⁺, IL7R⁻, LAG-3⁺, LAYN⁺, LEF1⁻, PD-1⁺, PRF1⁺, TCF7⁻, TIGIT⁺, TIM-3⁺, and TOX⁺; and
(b) any one or more of the following proteins: 4-1BB⁺, CCL3⁺, CD28⁻, CD39⁺, CD62L⁻ (SELL⁻), CD69⁺, CTLA4⁺, CX3CR1⁺, CXCL13⁺, CXCR6⁺, GZMA⁺, GZMB⁺, GZMK⁺, IL7R⁻, LAG-3⁺, LAYN⁺, LEF1⁻, PD-1⁺, PRF1⁺, TCF7⁻, TIGIT⁺, TIM-3⁺, and TOX⁺.

56. The method of claim 36, further comprising screening the tumor for expression of the tumor-specific antigen.

57. The method of claim 36, comprising administering no more than a single dose of the vaccine to the mammal.

58. The method of claim 36, comprising administering two, three, or more doses of the vaccine to the mammal.

59. The method of claim 58, comprising administering the vaccine to the mammal every other day starting on a first day that the T cells are administered to the mammal.

60. The method of claim 36, wherein the isolated T cells express any one or more of the following markers of differentiation:
(a) RNA encoding any one or more of: CCR7⁻, CD27⁻, CD45RA⁺, CD45RO⁻, CD95⁺, EOMES⁻, FOXO1⁻, KLRG1⁺, T-BET⁺, TCF7⁻, TOX⁺, and ZEB2⁺; and
(b) any one or more of the following proteins: CCR7⁻, CD27⁻, CD45RA⁺, CD45RO⁻, CD95⁺, EOMES⁻, FOXO1⁻, KLRG1⁺, T-BET⁺, TCF7⁻, TOX⁺, and ZEB2⁺.

61. The method of claim 36, comprising administering the vaccine to the mammal intramuscularly, subcutaneously, intravenously, or intraperitoneally.

62. The method of claim 36, comprising administering the T cells to the mammal intravenously or intraperitoneally.

63. The method of claim 36, wherein the isolated T cells are CD4⁺.

64. The method of claim 36, wherein the isolated T cells are CD8⁺.

65. The method of claim 36, wherein the mammal is a human.

66. The method of claim 36, wherein (i) and (ii) are administered to the mammal within 24 hours of each other.

67. The method of claim 36, further comprising administering an adjuvant to the mammal.

68. The method of claim 67, wherein the adjuvant comprises an anti-CD40 antibody or an anti-PD-1 antibody.

69. The method of claim 36, wherein the tumor-specific neoantigen is a personal neoantigen encoded by one or more somatic mutation(s) that are unique to the mammal's tumor, optionally wherein the tumor-specific neoantigen is not a tumor-specific driver mutation.

70. The method of claim 36, wherein the isolated T cells are terminally differentiated.
